(19) United States
(12) Patent Application Publication
(10) Pub. No.: US 2025/0134883 A1
(43) Pub. Date: May 1, 2025

(54) NOVEL COMPOSITIONS

(71) Applicant: TULEX PHARMACEUTICALS INC., Cranbury, NJ (US)

(72) Inventors: Yu-Hsing TU, Cranbury, NJ (US); Yingjun FAN, Plainsboro, NJ (US); Kalyan Kumar KATHALA, Monroe, NJ (US); Ashok Perumal, Monmouth Junction, NJ (US); Shanmuka Harish CHALAMURI, Plainsboro, NJ (US)

(21) Appl. No.: 18/833,346

(22) PCT Filed: Jan. 26, 2023
(86) PCT No.: PCT/US2023/061408

§ 371 Date: Jul. 25, 2024

Related U.S. Application Data
(60) Provisional application No. 63/303,354, filed on Jan. 26, 2022.

(51) Int. Cl. A61K 31/485; A61K 9/00; A61K 9/50; A61K 47/12; A61K 47/14; A61K 47/18; A61K 47/26; A61K 47/36

(52) U.S. Cl. CPC A61K 31/485; A61K 9/0053; A61K 9/5031; A61K 9/5042; A61K 9/5047; A61K 9/5089; A61K 47/12; A61K 47/14; A61K 47/183; A61K 47/26; A61K 47/36

(57) ABSTRACT

The present disclosure is directed to coated drug ion exchange particles and granules containing a resinous core, one or more active pharmaceutical agents and a coating comprising a cellulose acetate polymer optionally in combination with an enteric polymer. Related compositions and methods of making are disclosed.

### CROSS REFERENCE TO RELATED APPLICATIONS

This application claims priority benefit of U.S. Provisional Application No. 63/303,354 filed Jan. 26, 2022, the entire contents of which are incorporated by reference herein.

### FIELD OF THE DISCLOSURE

The present disclosure generally relates to novel coated drug-ion exchange resin particles (also referred to as coated drug-ion exchange resin granules) and pharmaceutical compositions containing such particles. More specifically, the disclosure provides compositions which are designed to provide an extended release of drug over time after administration.

### BACKGROUND OF THE DISCLOSURE

Extended-release pharmaceutical formulations, which release drug over a period of time, are widely used in the pharmaceutical industry. Such formulations provide several potential advantages to the patient including: (1) the convenience of reduced dosing frequency, (2) optimization of therapy by providing a smoother, more constant, plasma level of drug, and (3) a potential reduction in side effects.

Use of ion-exchange resins to form a drug-ion exchange resin complex is also known in the art. However, these drug-ion exchange resin complexes are subject to several drawbacks. For example, an ion exchange resins have been used to form a complex with ionic drugs to delay the drug release from such complexes is described. However, in many cases such delay in drug release has only been for a short duration.

Sustained or prolonged release dosage forms of various drugs are known and commercially available. However, providing a coating for sustained release of a drug from the fine particles of coated drug-ion exchange complexes has proven difficult. For example, various compositions of the art require time consuming steps for manufacture, as well as carefully monitored processing parameters with sophisticated equipment. Additionally, manufacture may include the use of a potentially hazardous step of coating from a solvent-based solution, which must be thoroughly removed from the pharmaceutical products before ingestion.

Various prior art references suggest treating drug-ion exchange resin complexes with water soluble, hydrophilic impregnating (solvating) agents such as polyethylene glycol and others to provide the coating with a water-permeable diffusion barrier. However, these agents cause the drug-ion exchange resin to swell when in contact with water, leading the coating layer to fracture and prematurely release the drug.

Similarly, there have been drawbacks associated with previously used polymers of acrylate and methacrylate-based aqueous dispersion coating systems for coating drug-ion exchange resin complex. Amongst these shortcomings observed is significant tackiness upon application of the coating and during curing, which complicates the coating process of drug-ion exchange resin complexes and/or requires the addition of further components such as an anti-tacking material to counteract this undesirable property.

A major risk of a sustained release dosage forms is its potential to release a drug more rapidly than intended. Such rapid drug release from a sustained release dosage form results in the administration of a single bolus dose leading to increased exposure levels, possible safety issues and adverse events. For example, alcohol is known to disrupt drug release for many extended-release formulations, leading to the entire dose being released at once due to ingestion of ethanol. Thus, alcohol induced dose dumping (AIDD) can be defined as the rapid unintended release of a large amount of drug from extended-release composition resulting from accidental misuse or intentional abuse of alcohol with the drug. Co-consumption with alcohol can additionally complicate matters because it may influence the absorption, metabolism and/or excretion of drugs.

Thus, new coated drug-ion exchange resin particles and compositions containing them are necessary to overcome the issues of the prior art. It would thus be beneficial to provide coated drug resin exchange particles and compositions containing the same which are capable of being efficiently and safely manufactured, and capable of providing extended release of one or more drugs. It would also be beneficial to provide such particles and compositions which can be used with a variety of drugs, and which are tunable to release the drugs at certain rates. It would be further beneficial to provide particles and compositions which are resistant to drug dumping, e.g., alcohol induced drug dumping.

Naltrexone, a prescription drug with brand names Revia® and Vivitrol® among others, is an oral opioid receptor antagonist approved by the US Food and Drug Administration (FDA) in 1984 for opiate addiction and in 1994 for alcohol dependence at doses of 50-100 mg/day. High dose naltrexone (0.5, 1.0 and 2.0 mg/kg) has been shown to reverse autistic behavior and gaze aversion (Lensing et al., Neuropsychobiology. 1995; 31:16-23). And a reduction in opioid tone in autism spectrum disorder (ASD) patients correlated with treatment response (Cazullo et al., Eur. Neuropsychopharmacol. 1999; 9:361-6).

However, given the side effects of naltrexone treatments, there is a need in the art to provide a naltrexone formulation that mitigates the side effects associated with naltrexone treatments. The present disclosure provides a naltrexone formulation that addresses these needs.

### SUMMARY OF THE DISCLOSURE

Provided herein are coated drug resin exchange particles (or granules) (“coated granules” or “coated particles”) and compositions containing them that overcome the issues of the prior art. The coated granules and compositions of the present disclosure are capable of being efficiently and safely manufactured, and are also capable of providing extended release of one or more drugs. The coated granules and compositions of the present disclosure can be used with a variety of drugs, and are tunable to release the drugs at certain rates. Further, the coated granules and compositions of the present disclosure are resistant to drug dumping, e.g., alcohol induced drug dumping.

In a first embodiment, the present disclosure provides a coated drug-ion exchange resin particle (or coated drug-ion exchange resin granule) [Coated Granule 1] comprising:
a) a drug-ion exchange resin complex (or resinous core) comprising:
   an ion exchange resin;
   one or more active pharmaceutical agents;
   optionally a chelating agent;
   optionally a granulating agent; and
b) a coating comprising:
   a cellulose acetate-containing polymer;
   optionally, an enteric polymer; and
   optionally, a plasticizer.

In some embodiments, the ion exchange resin is, e.g., a polystyrene polymer. In some embodiments, the cellulose acetate-containing polymer is selected from the group consisting of cellulose acetate, cellulose diacetate, cellulose triacetate, cellulose acetate butyrate, cellulose acetate phthalate (CAP), cellulose acetate propionate, and combinations thereof. In some further aspects, the enteric polymer is selected from: a phthalate-containing polymer, e.g., cellulose acetate phthalate (CAP), hypromellose phthalate (hydroxypropylmethylcellulose phthalate; HPMCP), polyvinyl acetate phthalate (PVAP); cellulose acetate trimellitate; polymethacrylates (e.g., Eudragit® L series, Eudragit® S series, Eudragit® FS, Kollicoat® MAE 100P, Acryl-EZE® 93A, Acryl-EZE® MP); hydroxypropylmethylcellulose acetate succinate (HPMC AS; Aqoat®); sodium alginate; alginic acid, shellac and combinations thereof. In some embodiments, the enteric polymer can also be a cellulose acetate-containing polymer preferably cellulose acetate phthalate (CAP). In some preferred embodiments, the ion exchange resin comprises a polystyrene polymer; the chelating agent is present and comprises EDTA, citric acid or a combination of EDTA and citric acid; the granulating agent is present and comprises polyethylene glycol; and the plasticizer is present and comprises polyethylene glycol.

In some embodiments, the present disclosure provides a composition for sustained release of an active pharmaceutical agent comprising the coated drug-ion exchange resin particle (or coated drug-ion exchange resin granule) as described herein, and a pharmaceutically acceptable excipient. In some preferred embodiments, the composition is a liquid suspension comprising the Coated Granules, one or more solvents, and one or more ingredients selected from sugars, antioxidants, chelating agents, thickeners, stabilizers, preservatives, surfactants, flavorings and coloring agents. In some preferred embodiments, the composition is a liquid suspension comprising a sugar; one or more chelating agents; one or more thickeners; a preservative; a surfactant; and an antioxidant.

In some further embodiments, the present disclosure provides methods of making the particles and compositions described herein.

### BRIEF DESCRIPTION OF THE FIGURES

FIG. 1 shows the in-vitro drug release profile for the Naltrexone ER Suspension formulation described in Example 2, Table 5 herein.

FIG. 2 shows drug release profiles of coated naltrexone resin particles having different polymer coatings. In the Figure, CAB is cellulose acetate butyrate coated particle formulation of Table 3, CAB and HPMCP (Hypromellose phthalate) formulation of Table 6, and EC (ethyl cellulose) formulation of Table 7.

FIG. 3 shows the in vitro drug release profiles for Naltrexone ER Suspension Formulations 1 and 2 described in Table 16.

FIG. 4 shows the in-vivo naltrexone drug release kinetics for Formulations 1 and 2, and 50 mg Naltrexone HCl Tablets, described in Table 18.

FIG. 5 shows the in-vivo 6-β naltrexol drug release kinetics for Formulations 1 and 2, and 50 mg Naltrexone HCl Tablets, described in Table 19.

### DETAILED DESCRIPTION OF THE DISCLOSURE

In a first aspect, the present disclosure provides a coated drug-ion exchange resin particle (or coated drug-ion exchange resin granule) [Coated Granule 1] comprising:
c) a drug-ion exchange resin complex (or resinous core) comprising:
   an ion exchange resin;
   one or more active pharmaceutical agents;
   optionally a chelating agent;
   optionally a granulating agent; and
d) a coating comprising:
   a cellulose acetate-containing polymer;
   optionally, an enteric polymer; and
   optionally, a plasticizer.

The present disclosure further provides the following embodiments of Coated Granule 1:
1.1 Coated Granule 1, wherein the resinous core comprises a polystyrene polymer.
1.2 Coated Granule 1 or 1.1, wherein the ion exchange resin comprises a co-polymer of polystyrene and divinylbenzene.
1.3 Any of the preceding Coated Granules, wherein the ion exchange resin comprises matrix of sulfonated polymers composed of polystyrene cross-linked with about 8% of divinylbenzene
1.4 Any of the preceding Coated Granules, wherein the ion exchange resin has an ion-exchange capacity of about 4.5 to 5.5 meq/g of dry resin.
1.5 Any of the preceding Coated Granules, wherein the resinous core comprises a particle size of about 45 microns to 150 microns.
1.6 Any of the preceding Coated Granules, wherein the resinous core comprises particles which are spherical or irregularly shaped.
1.7 Any of the preceding Coated Granules, wherein the ion exchange resin comprises Amberlite IRP-69.
1.8 Any of the preceding Coated Granules, wherein the ion exchange resin and the one or more active pharmaceutical agents are present in a w/w ratio of about 5:1 to 1:5, e.g., about 3:1 to 1:3, e.g., about 2:1 to 1:2, e.g., about 2:1.
1.9 Any of the preceding Coated Granules, wherein the cellulose acetate-containing polymer is selected from cellulose acetate, cellulose diacetate, cellulose triacetate, cellulose acetate butyrate, cellulose acetate phthalate, cellulose acetate propionate, and combinations thereof.
1.10 Any of the preceding Coated Granules, wherein the cellulose acetate-containing polymer is selected from cellulose acetate, cellulose acetate butyrate, and combinations thereof.
1.11 Any of the preceding Coated Granules, wherein the cellulose acetate-containing polymer comprises cellulose acetate.
1.12 Any of the preceding Coated Granules, wherein the cellulose acetate-containing polymer consists essentially of, or consists of cellulose acetate.
1.13 Any of the preceding Coated Granules, wherein the cellulose acetate-containing polymer comprises cellulose acetate butyrate.
1.14 Any of the preceding Coated Granules, wherein the cellulose acetate-containing polymer consists essentially of, or consists of cellulose acetate butyrate.
1.15 Any of the preceding Coated Granules, wherein the enteric polymer is selected from a phthalate-containing polymer; cellulose acetate trimellitate; polymethacrylates; hydroxypropylmethylcellulose acetate succinate (HPMC AS; Aqoat®); sodium alginate; alginic acid; shellac and combinations thereof.
1.16 Any of the preceding Coated Granules, wherein the enteric polymer is or comprises a phthalate-containing polymer comprising monomers of dimethyl phthalate, diethyl phthalate, diallyl phthalate, di-n-propyl phthalate, di-n-butyl phthalate, diisobutyl phthalate, butyl cyclohexyl phthalate, di-n-pentyl phthalate, dicylcohexyl phthalate, butyl benzyl phthalate, di-n-hexyl phthalate, diisohexyl phthalate, diisoheptyl phthalate, butyl decyl phthalate, dibutoxy ethyl phthalate, di(2-ethylhexyl) phthalate, di(n-octyl) phthalate, diisooctyl phthalate, n-octyl n-decyl phthalate, diisononyl phthalate, di(2-propylheptyl) phthalate, diisodecyl phthalate, diundecyl phthalate, diisoundecyl phthalate, ditridecyl phthalate, diisotridecyl phthalate, polyethylene terephthalate, cellulose phthalate, cellulose acetate phthalate (CAP), hydroxypropylmethylcellulose phthalate (HPMCP), polyvinyl acetate phthalate (PVAP) and combinations thereof.
1.17 Any of the preceding Coated Granules, wherein the enteric polymer comprises a phthalate-containing polymer selected from cellulose acetate phthalate (CAP), hypromellose phthalate (hydroxypropylmethylcellulose phthalate; HPMCP), and polyvinyl acetate phthalate (PVAP).
1.18 Any of the preceding Coated Granules, wherein the enteric polymer is a phthalate-containing polymer selected from cellulose acetate phthalate (CAP), hypromellose phthalate (hydroxypropylmethylcellulose phthalate; HPMCP), and polyvinyl acetate phthalate (PVAP).
1.19 Any of the preceding Coated Granules, wherein the enteric polymer comprises cellulose acetate trimellitate.
1.20 Any of the preceding Coated Granules, wherein the enteric polymer is cellulose acetate trimellitate.
1.21 Any of the preceding Coated Granules, wherein the enteric polymer comprises a polymethacrylate.
1.22 Any of the preceding Coated Granules, wherein the enteric polymer is a polymethacrylate.
1.23 Any of the preceding Coated Granules, wherein the enteric polymer comprises a polymethacrylate selected from Eudragit® L series, Eudragit® S series, Eudragit® FS, Kollicoat® MAE 100P, Acryl-EZE® 93A, and Acryl-EZER MP.
1.24 Any of the preceding Coated Granules, wherein the enteric polymer is a polymethacrylate selected from Eudragit® L series, Eudragit® S series, Eudragit® FS, Kollicoat® MAE 100P, Acryl-EZER 93A, or Acryl-EZER MP.
1.25 Any of the preceding Coated Granules, wherein the enteric polymer comprises hydroxypropylmethylcellulose acetate succinate (HPMC AS; Aqoat®).
1.26 Any of the preceding Coated Granules, wherein the enteric polymer is hydroxypropylmethylcellulose acetate succinate (HPMC AS; Aqoat®).
1.27 Any of the preceding Coated Granules, wherein the enteric polymer comprises sodium alginate or alginic acid.
1.28 Any of the preceding Coated Granules, wherein the enteric polymer is sodium alginate or alginic acid.
1.29 Any of the preceding Coated Granules, wherein the enteric polymer comprises shellac.
1.30 Any of the preceding Coated Granules, wherein the enteric polymer is shellac.
1.31 Any of the preceding Coated Granules, further comprising an anti-tacking agent; for example and not limited to an anti-tacking agent such as talc, glycerol monostearate, magnesium stearate or kaolin.
1.32 Any of the preceding Coated Granules, wherein the granulating agent is present.
1.33 Any of the preceding Coated Granules, wherein the granulating agent in the drug-ion exchange resin complex is selected from benzophenone, butyl phthalyl butyl glycollate, camphor, alpha-cresyl-p-toluene sulfonate, cyclohexyl-p-toluene sulfonamide, diamyl phthalate, dibutyl phthalate, dibutyl sebacate, dibutyl succinate, dibutyl tartrate, diethoxyethyl adipate, diethoxyethyl phthalate, diethyl adipate, diethylene glycol dipropionate, diethyl phthalate, diethyl sebacate, diethyl succinate, diethyl tartrate, dimethoxyethyl adipate, dimethoxyethyl phthalate, dimethyl phthalate, dipropyl phthalate, ethyl benzoyl benzoate, ethylene glycol diacetate, ethylene glycol dibutyrate, ethylene glycol dipropionate, ethyl phthalyl ethyl glycolate, methyl benzoyl benzoate, methyl phthalyl ethyl glycolate, o- or p-toluene ethyl sulfonamide, triacetin, tributyl citrate, tributyl phosphate, tributyrin, tricresyl phosphate, triethylene glycol diacetate, triethylene glycol dibutyrate, triethylene glycol diproprionate, triphenyl phosphate, tripropionin, trimellitates (e.g., tri-(2-ethylhexyl) trimellitate, tri-(isononyl) trimellitate, tri-(isodecyl) trimellitate, tri-(isotridecyl) trimellitate), adipates (e.g., bis(2-ethylhexyl) adipate), sebacates (e.g., dibutyl sebacate, di-(2-ethylhexyl) sebacate), glycerol triacetate, alkyl citrates (triethyl citrate, acetyl triethyl citrate, tributyl citrate, acetyl trybutyl citrate, tri (2-ethylhexyl) citrate, acetyl trioctyl citrate, trihexyl citrate, butyryl trihexyl citrate), vegetable oils, epoxidized soybean oil, epoxidized linoleic oil, azelates, dibenzoates, terephthalates, 1,2-cyclohexane dicarboxylic acid diisononyl ester, alkyl sulphonic acid phenyl ester, organophosphates (e.g., tricresyl (methyl phenyl) phosphate, 2-ethylhexyldiphenyl phosphate), glycols (e.g., propylene glycol, polyethylene glycol, triethylene glycol di-2hexanoate) triacetin, triethyl citrate, dibutyl sebacate, vegetable oil, lipids and combinations thereof.
1.34 Any of the preceding Coated Granules, wherein the granulating agent in the drug-ion exchange resin complex comprises polyethylene glycol; for example polyethylene glycol 3350.
1.35 Any of the preceding Coated Granules, wherein the granulating agent in the drug-ion exchange resin complex consists of polyethylene glycol; for example polyethylene glycol 3350.
1.36 Any of the preceding Coated Granules, wherein the plasticizer is present.
1.37 Any of the preceding Coated Granules, wherein the plasticizer is selected from benzophenone, butyl phthalyl butyl glycollate, camphor, alpha-cresyl-p-toluene sulfonate, cyclohexyl-p-toluene sulfonamide, diamyl phthalate, dibutyl phthalate, dibutyl sebacate, dibutyl succinate, dibutyl tartrate, diethoxyethyl adipate, diethoxyethyl phthalate, diethyl adipate, diethylene glycol dipropionate, diethyl phthalate, diethyl sebacate, diethyl succinate, diethyl tartrate, dimethoxyethyl adipate, dimethoxyethyl phthalate, dimethyl phthalate, dipropyl phthalate, ethyl benzoyl benzoate, ethylene glycol diacetate, ethylene glycol dibutyrate, ethylene glycol dipropionate, ethyl phthalyl ethyl glycolate, methyl benzoyl benzoate, methyl phthalyl ethyl glycolate, o- or p-toluene ethyl sulfonamide, triacetin, tributyl citrate, tributyl phosphate, tributyrin, tricresyl phosphate, triethylene glycol diacetate, triethylene glycol dibutyrate, triethylene glycol diproprionate, triphenyl phosphate, tripropionin, trimellitates (e.g., tri-(2-ethylhexyl) trimellitate, tri-(isononyl) trimellitate, tri-(isodecyl) trimellitate, tri-(isotridecyl) trimellitate), adipates (e.g., bis(2-ethylhexyl) adipate), sebacates (e.g., dibutyl sebacate, di-(2-ethylhexyl) sebacate), glycerol triacetate, alkyl citrates (triethyl citrate, acetyl triethyl citrate, tributyl citrate, acetyl trybutyl citrate, tri (2-ethylhexyl) citrate, acetyl trioctyl citrate, trihexyl citrate, butryyl trihexyl citrate), vegetable oils, epoxidized soybean oil, epoxidized linoleic oil, azelates, dibenzoates, terephthalates, 1,2-cyclohexane dicarboxylic acid diisononyl ester, alkyl sulphonic acid phenyl ester, organophosphates (e.g., tricresyl (methyl phenyl) phosphate, 2-ethylhexyldiphenyl phosphate), glycols (e.g., propylene glycol, polyethylene glycol, triethylene glycol di-2hexanoate) triacetin, triethyl citrate, dibutyl sebacate, vegetable oil, lipids and combinations thereof.
1.38 Any of the preceding Coated Granules, wherein the plasticizer is selected from propylene glycol, polyethylene glycol, triacetin, triethyl citrate, acetyl triethyl citrate, dibutyl sebacate, acetyl tributyl citrate, tributyl citrate, Soluphor P, vegetable oil, lipids, and combinations thereof.
1.39 Any of the preceding Coated Granules, wherein the plasticizer in the coating comprises polyethylene glycol; for example polyethylene glycol 3350.
1.40 Any of the preceding Coated Granules, wherein the plasticizer in the coating consists of polyethylene glycol; for example polyethylene glycol 3350.
1.41 Any of the preceding Coated Granules, wherein the plasticizer is dispersed throughout the coating.
1.42 Any of the preceding Coated Granules, wherein the plasticizer comprises about 2.5 to about 25% w/w, or about 5 to about 15% w/w, or about 8 to about 12% w/w, or about 10% w/w of the solids in the coating of solids in the coating.
1.43 Any of the preceding Coated Granules, further comprising one or more additional active pharmaceutical agents, e.g., 2-10 active pharmaceutical agents.
1.44 Any of the preceding Coated Granules, wherein the resinous core comprises one or more active pharmaceutical agents that are small molecule drugs, e.g., up to 10 small molecule drugs.
1.45 Any of the preceding Coated Granules, wherein the active pharmaceutical agent is a free base compound.
1.46 Any of the preceding Coated Granules, wherein the active pharmaceutical agent is a compound which may be protonated at one or more locations on the molecule.
1.47 Any of the preceding Coated Granules, wherein the active pharmaceutical agent is present in an amount of about 1% to about 99% by weight based on the total weight of the coated drug-ion exchange resin granule, from about 5% to about 70% by weight, from about 10% to about 30% by weight, from about 15% to about 25% by weight, or about 20% by weight based on the total weight of the coated drug ion exchange resin granule.
1.48 Any of the preceding Coated Granules, wherein the active pharmaceutical agent is released at a higher initial rate that tapers over a sustained period of time.
1.49 Any of the preceding Coated Granules, wherein the Coated Granule provides sustained release of the active pharmaceutical agent for at least 1 hour, at least 4 hours, at least 8 hours, at least 16 hours, or at least 24 hours.
1.50 Any of the preceding Coated Granules, wherein the Coated Granule provides sustained release of the active pharmaceutical agent for 8-24 hours.
1.51 Any of the preceding Coated Granules, wherein the Coated Granule provides sustained release of the active pharmaceutical agent for up to 24 hours.
1.52 Any of the preceding Coated Granules, wherein the Coated Granule is contained within a composition which is in the form of a suspension, capsule, sachet, tablet, chewable tablet, tablet for dispersion or orally disintegrating tablet (ODT).
1.53 Any the preceding Coated Granules, wherein the active pharmaceutical agent is dispersed (e.g., substantially homogenously dispersed): throughout the resinous core, e.g., within the resinous core and on the surface of the resinous core; and/or throughout the Coated Granule.
1.54 Any of the preceding Coated Granules, wherein the active pharmaceutical drug is bound to the resinous core (e.g., bound via covalent or ionic bonding).
1.55 Any of the preceding Coated Granules, wherein the active pharmaceutical drug is bound to the resinous core via ionic bonding.
1.56 Any of the preceding Coated Granules, wherein the coating comprises a mixture (e.g., a substantially homogenous mixture) of the cellulose acetate-containing polymer and the enteric polymer.
1.57 Any of the preceding Coated Granules, wherein the coating comprises a mixture (e.g., a substantially homogenous mixture) of cellulose acetate or cellulose acetate butyrate and the enteric polymer.
1.58 Any of the preceding Coated Granules, wherein the coating comprises a mixture (e.g., a substantially homogenous mixture) of cellulose acetate or cellulose acetate butyrate and one or more of cellulose acetate phthalate (CAP), hypromellose phthalate (hydroxypropylmethylcellulose phthalate; HPMCP), and polyvinyl acetate phthalate (PVAP).
1.59 Any of the preceding Coated Granules, wherein the cellulose acetate-containing polymer and the enteric polymer are present in a w/w ratio of about 5:1 to about 1:5.
1.60 Any of the preceding Coated Granules, wherein the cellulose acetate-containing polymer and the enteric polymer are present in a w/w ratio of about 2:1 to about 1:2.
1.61 Any of the preceding Coated Granules, wherein the cellulose acetate-containing polymer and the enteric polymer are present in a w/w ratio of about 1:1.
1.62 Any of the preceding Coated Granules, wherein the Coated Granule has a particle size of about 10 microns to about 500 microns, e.g., about 50 microns to about 250 microns, e.g., about 100 microns to about 410 microns, e.g., about 100 microns to about 250 microns, e.g. about 150-250 microns.
1.63 Any of the preceding Coated Granules, wherein the majority of the Coated Granules have a particle size of about 150-250 microns.
1.64 Any of the preceding Coated Granules, wherein the coated drug-ion exchange resin particle is spherical, polyhedral, or irregularly shaped.
1.65 Any of the preceding Coated Granules, wherein the coated drug-ion exchange resin particle is irregularly shaped.
1.66 Any of the preceding Coated Granules, wherein the coating is bound to an outer surface of the resinous core, e.g., and substantially covers the entirety of the outer surface.
1.67 Any of the preceding Coated Granules, wherein the coating comprises a substantially homogenous mixture of the cellulose acetate polymer alone or in combination with the enteric polymer, and is applied to and/or is present on an outer surface of the resinous core, e.g., the entirety of the outer surface.
1.68 Any of the preceding Coated Granules, wherein the coating comprises a substantially homogenous mixture of cellulose acetate or cellulose acetate butyrate and cellulose acetate phthalate, and is applied to and/or is present on an outer surface of the resinous core e.g., the entirety of the outer surface.
1.69 Any of the preceding Coated Granules, wherein the Coated Granules comprise the cellulose acetate-containing polymer and the enteric polymer.
1.70 Any of the preceding Coated Granules, wherein the coating comprises a mixture (e.g., a substantially homogenous mixture) of the cellulose acetate-containing polymer, e.g. cellulose acetate butyrate, and polyethylene glycol.
1.71 Any of the preceding Coated Granules, wherein the coating comprises a substantially homogenous mixture of cellulose acetate butyrate and polyethylene glycol, for example wherein the mixture optionally is applied to an outer surface of the drug-ion exchange resin complex granule.
1.72 Any of the preceding Coated Granules, wherein the active pharmaceutical agent is released at a rate of 20%-45% in two hours, and/or 40%-65% in four hours, and/or greater than 80% in twelve hours in 900 mL 0.4M KH₂PO₄ at 50 rpm, 37±0.5° C. using Apparatus II described in chapter <711> of United States Pharmacopeia (USP) 43; for example and not limited to wherein no more than 25% of the active pharmaceutical agent is released within one hour and wherein no more than 60% of the active pharmaceutical agent is released within two hours.
1.73 Any of the preceding Coated Granules, wherein the ion exchange resin comprises Amberlite IRP-69; the cellulose acetate-containing polymer comprises cellulose acetate, cellulose acetate butyrate or a combination thereof; and the enteric polymer comprises one or more of cellulose acetate phthalate (CAP), hypromellose phthalate (hydroxypropylmethylcellulose phthalate; HPMCP) and polyvinyl acetate phthalate (PVAP).
1.74 Any of the preceding Coated Granules, wherein the chelating agent is present.
1.75 Any of the preceding Coated Granules, wherein the granulating agent is present.
1.76 Any of the preceding Coated Granules, wherein the plasticizer is present.
1.77 Any of the preceding Coated Granules, wherein the chelating agent, granulating agent and plasticizer are present.
1.78 Coated Granule 1.77, wherein the chelating agent comprises or consists of EDTA; the granulating agent comprises or consists of polyethylene glycol, for example polyethylene glycol 3550; and the plasticizer comprises or consists of polyethylene glycol, for example polyethylene glycol 3550.
1.79 Any of the preceding Coated Granules, wherein:
   the ion exchange resin comprises Amberlite IRP69;
   the chelating agent is present and comprises EDTA;
   the granulating agent is present and comprises polyethylene glycol, for example polyethylene glycol 3550; and
   the plasticizer is present and comprises polyethylene glycol, for example polyethylene glycol 3550.
1.80 Any of the preceding Coated Granules, wherein the resinous core comprises from about 70% w/w to about 90% w/w of the Coated Granules; for example from about 75% w/w to about 90% w/w of the Coated Granules; for example from about 80% w/w to about 85% w/w of the Coated Granules; for example from about 83% w/w to about 84% w/w of the Coated Granules.
1.81 Any of the preceding Coated Granules, wherein the cellulose acetate-containing polymer comprises from about 5% w/w to about 25% w/w of the Coated Granules; for example from about 10% w/w to about 20% w/w of the Coated Granules; for example from about 12% w/w to about 18% w/w of the Coated Granules; for example from about 14% w/w to about 16% w/w of the Coated Granules; for example, about 15% w/w of the Coated Granules.
1.82 Any of the preceding Coated Granules, wherein the granulating agent comprises from about 0.5% w/w to about 5% w/w of the Coated Granules; for example from about 0.5% w/w to about 3% w/w of the Coated Granules; for example from about 1% w/w to about 2% w/w of the Coated Granules; for example about 1.5% w/w of the Coated Granules.
1.83 Any of the preceding Coated Granules, having the following composition:

| | |
|---|---|
| Ingredient | % w/w |

Coated drug (e.g. naltrexone)-ion exchange resin complex granules

| | |
|---|---|
| Drug-ion exchange resin granules (or | 70-90; e.g. 75-90; e.g. 80- |
| resinous core); e.g., an uncoated | 85; e.g. 83-84; e.g. 83.3 |
| naltrexone-ion exchange resin complex | |
| Cellulose acetate-containing polymer, | 5-25; e.g. 10-20; e.g. 12- |
| e.g. cellulose acetate butyrate | 18; e.g. 14-16%; e.g. 15- |
| | 15.4; e.g. 15.2 |
| Plasticizer, e.g. polyethylene glycol | 0.5-5; e.g. 0.5-3; e.g. 1-2; |
| (PEG), e.g. PEG 3350 | e.g. 1.5 |

1.84 Any of the preceding Coated Granules, wherein the active pharmaceutical agent comprises naltrexone or a pharmaceutically acceptable salt thereof.
1.85 Any of the preceding Coated Granules, wherein the drug-ion exchange resin complex granule comprises a naltrexone-ion exchange resin complex combined with (e.g. bound to) the chelating agent and the granulating agent.
1.86 Coated Granule 1.83, wherein the naltrexone-ion exchange resin complex comprises the ion exchange resin and the naltrexone or a pharmaceutically acceptable salt thereof.

The present disclosure further provides a composition comprising a plurality of any of the preceding Coated Granules 1 or 1.1-1.86. For example, in a second aspect, the present disclosure provides a composition [Composition 1] for sustained release of an active pharmaceutical agent, said composition comprising:
a coated drug-ion exchange resin particle (or granule) according to any of Coated Granules 1 or 1.1-1.86; and
a pharmaceutically acceptable excipient.

The present disclosure further provides the following embodiments of Composition 1:
1.1 Composition 1, wherein the Composition is in the form of a suspension, capsule, sachet, tablet, chewable tablet, tablet for dispersion or orally disintegrating tablet (ODT).
1.2 Composition 1 or 1.1, wherein the Composition is in the form of a liquid suspension.
1.3 Any of the preceding Compositions, wherein the composition is a suspension comprising the Coated Granules, one or more solvents, and one or more ingredients selected from sugars, antioxidants, chelating agents, buffering agents, thickeners, stabilizers, preservatives, surfactants, flavorings and coloring agents.
1.4 Any of the preceding Compositions, wherein the solvent in the suspension comprises water.
1.5 Any of the preceding Compositions, wherein the suspension comprises the Coated Granules in an amount of from about 0.01 g to about 0.03 g of Coated Granules per ml of suspension.
1.6 Any of the preceding Compositions, wherein the Coated Granules are present in the composition in an amount of about 0.01% w/v to about 10% w/v, based on the total weight of the composition; or about 0.01% w/v to about 10% w/v, based on the total weight of the composition; or about 0.1% w/v to about 5% w/v, based on the total weight of the composition; or about 0.1% w/v to about 5% w/v, based on the total weight of the composition; or about 1% w/v to about 5% w/v, based on the total weight of the composition; or about 1% w/v to about 5% w/v, based on the total weight of the composition; or about 2% w/v to about 3% w/v, based on the total weight of the composition; or about 2% w/v to about 3% w/v, based on the total weight of the composition.
1.7 Any of the preceding Compositions, wherein the pharmaceutically acceptable excipient comprises one or more of a carrier, diluents, thickeners, binders, lubricants, humectants, disintegrants, anti-oxidants, surfactants, colorants, flavorants, preservatives, sweeteners, buffers, and combinations thereof.
1.8 Any of the preceding Compositions, wherein the composition is in the form of a suspension, for example a liquid suspension, and the active pharmaceutical agent is present in an amount from about 0.1 mg/mL to about 100 mg/mL, e.g., from about 1 mg/mL to about 25 mg/mL, e.g., from about 3 mg/mL to about 8 mg/mL, e.g., about 5 mg/mL.
1.9 Any of the preceding Compositions, wherein the composition is in the form of a suspension, for example a liquid suspension, and the active pharmaceutical agent is present in an amount from about 0.1% w/v to about 2% w/v, e.g., from about 0.1% to about 1% w/v; e.g., from about 0.2% w/v to about 0.8% w/v, e.g., from about 0.4% w/v to about 0.6% w/v, e.g. about 0.5% w/v, e.g. 0.49%-0.51% w/v.
1.10 Any of the preceding Compositions, wherein the Composition comprises a plurality of said Coated Granules.
1.11 Any of the preceding Compositions, wherein the Coated Granules are present in an amount of from 0.5% to about 5% w/v; for example from about 1.0% to about 3.0% w/v; for example from 2.0% to about 2.5% w/v, for example about 2.3% w/v.
1.12 Any of the preceding Compositions, wherein the Composition comprises a chelating agent, for example EDTA.
1.13 Any of the preceding Compositions, wherein the composition comprises a buffering agent, for example citric acid, for example citric acid anhydrous.
1.14 Composition 1.13, wherein the chelating agent, for example EDTA is present in an amount of from about 0.05% to about 0.15% w/v; for example from about 0.08% to about 0.12% w/v; for example from about 0.09% to about 0.10% w/v; for example about 0.095% w/v; and/or the buffering agent, e.g. citric acid, is present in an amount of from about 0.1% to about 0.8% w/v; for example from about 0.2% to about 0.6% w/v; for example from about 0.3% to about 0.5% w/v; for example about 0.4% w/v.
1.15 Any of the preceding Compositions, wherein the Composition comprises an antioxidant, for example propyl gallate, in an amount of from about 0.01% to about 0.03%, for example about 0.02%.
1.16 Any preceding Composition, wherein the Composition comprises a surfactant, for example Tween 80, in an amount of from about 0.01% to about 0.3% w/v; for example from about 0.05% to about 0.2% w/v; for example about 0.1% w/v.
1.17 Any preceding Composition, wherein the Composition comprises a solvent, for example water.
1.18 Any preceding Composition, wherein the Composition comprises a sugar, for example sucrose, in an amount of from about 5% to about 40% w/v; for example, from about 10% to about 30% w/v; for example, from about 15% to about 25% w/v; for example, about 20% w/v.
1.19 Any preceding Composition, wherein the Composition comprises a preservative, for example sodium benzoate, in an amount of from about 0.1% to about 0.5% w/v; for example from about 0.15% to about 0.3% w/v; for example about 0.2% w/v.
1.20 Any preceding Composition, wherein the Composition comprises a thickener, for example starch, xanthan gum, or a combination of starch and xanthan gum.
1.21 Composition 1.20 wherein the composition comprises both starch and xanthan gum.
1.22 Composition 1.21, wherein the starch is present in an amount of from about 0.1% to about 5% w/v; for example from about 1% to about 3% w/v; for example about 2% w/v; and the xanthan gum is present in an amount of from about 0.05% to about 0.5% w/v; for example from about 0.1% to about 0.2% w/v; for example about 0.15% w/v.
1.23 Any of the preceding Compositions, wherein the Composition comprises:
   a Coated Granule according to any of Coated Granules 1 or 1.1-1.84;
   a sugar (e.g. sucrose);
   a chelating agent (e.g. EDTA);
   a buffering agent (e.g., citric acid);
   one or more thickeners (e.g. starch and/or Xanthan gum);
   a preservative (e.g. sodium benzoate);
   a surfactant (e.g. TWEEN 80); and
   an antioxidant (e.g. propyl gallate).
1.24 Any preceding Composition, wherein the Coated Granule is a Coated Granule according to Coated Granule 1.79.
1.25 Composition 1.23 or 1.24, further comprising one or more coloring agents and/or flavor agents.
1.26 Composition 1.25, wherein the Composition is a liquid suspension.
1.27 Any of the preceding Compositions, wherein the Composition is in the form of a liquid suspension, and:
   the solvent comprises water;
   the sugar comprises sucrose;
   chelating agent comprises EDTA;
   the buffering agent comprises citric acid, e.g. anhydrous citric acid;
   the antioxidant is present and comprises propyl gallate;
   the thickener comprises one or more of starch and xanthan gum; and
   the surfactant comprises Tween 80.
1.28 Any of the preceding Compositions, having the following composition:

| Ingredient | Quantity (g) | % w/v |
|---|---|---|
| Sugar (e.g. Sucrose) | 600-1000; e.g. 800 | 5-40; e.g. 10- |
| | | 30%; e.g. 15%- |
| | | 25%; e.g. 20 |
| Buffering agent (e.g. citric acid | 10-20; e.g. 14-18; e.g. 16 | 0.1-0.8; e.g. 0.2- |
| anhydrous) | | 0.6; e.g. 0.25-0.5; |
| | | e.g. 0.3-0.5; e.g. |
| | | 0.35-0.45; e.g. 0.4 |
| Chelating agent (e.g. EDTA) | 2-6; e.g. 3-5; e.g. 3.5-4.5; e.g., 3.8 | 0.05-0.15; e.g. |
| | | 0.075-0.125; e.g. |
| | | 0.08-0.12; e.g. |
| | | 0.09-0.10; e.g. |
| | | 0.095 |
| Thickener (e.g. Starch) | 70-100; e.g. 80-90; e.g. 84-85, e.g. | 0.1-5; e.g. 1-3; |
| | 84.39 | e.g. 1.75-2.5; e.g. |
| | | 2.0-2.25; e.g. 2.1- |
| | | 2.3; e.g. 2; e.g., |
| | | 2.1 |
| Preservative (e.g. Sodium | 4-12; e.g. 6-10; e.g. 8 | 0.1-0.5; e.g. 0.1- |
| benzoate) | | 0.3; e.g. 0.15- |
| | | 0.25; e.g.0.2 |
| Thickener (e.g. Xanthan gum) | 2-10; e.g. 4-8; e.g. 5-7; e.g. 6 | 0.05-0.5; e.g. 0.1- |
| | | 0.2; e.g. 0.15 |
| Surfactant (e.g. Tween 80) | 2-6; e.g. 3-5; e.g. 4 | 0.01-0.3; e.g. |
| | | 0.05- 0.2; e.g. 0.1 |
| Anti-oxidant (e.g. propyl | 0.4-1.2; e.g. 0.6-1.0; e.g. 0.8 | 0.01-0.03; e.g. |
| gallate) | | 0.02 |
| Coated drug ion-exchange | 60-110; e.g. 70-100; e.g., 80-90; e.g. | 0.5-5; e.g. 1.0- |
| Granules | 85; e.g., 89 | 3.0; e.g. 2.0-2.5; |
| | | e.g. 2.3; e.g. about |
| | | 2.28 |
| Color agent (e.g. FD & C red | 0.1-1.3; e.g. 0.2 | 0.005 |
| 40) | | |
| Flavor (e.g. Cherry flavor) | 2-6; e.g. 4 | 0.1 |
| Purified water | QS to 4 L | QS to 4 L |

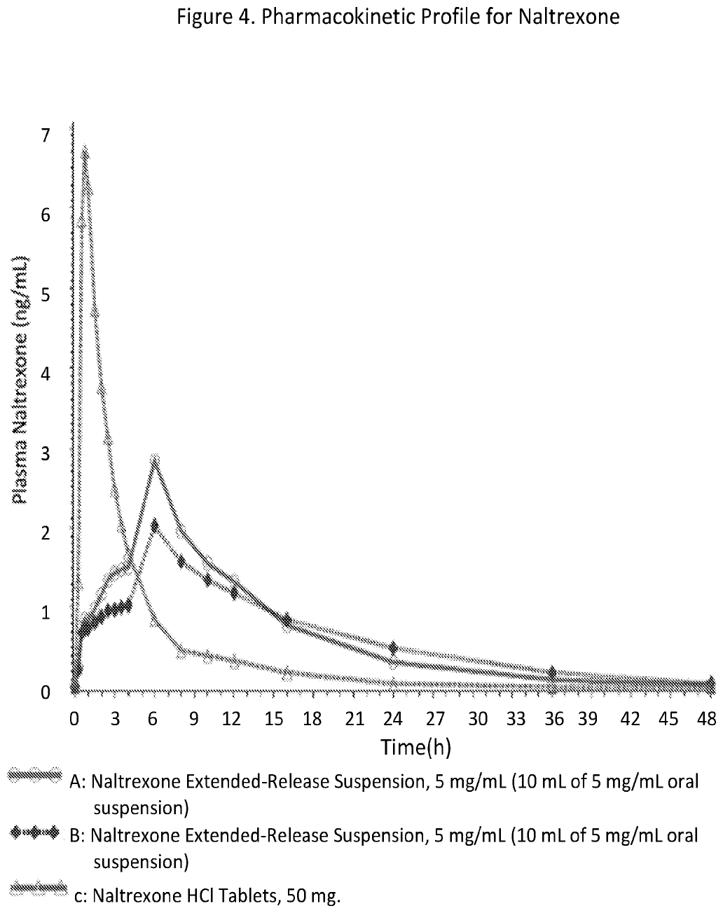

1.29 Any of the preceding Compositions, wherein the composition is a liquid suspension, and the suspension does not contain glycerin, or polyethylene glycol, other than in the Coated Granules.
1.30 Any of the preceding Compositions, wherein the active pharmaceutical agent is released at a higher initial rate that tapers over a sustained period of time.
1.31 Any of the preceding Particles, wherein the active pharmaceutical agent is released at a higher initial rate that tapers over a sustained period of time.
1.32 Any of the preceding Compositions, wherein the active pharmaceutical agent is released over a sustained period of time.
1.33 Any of the preceding Compositions, wherein the drug ion exchange particle provides sustained release of the active pharmaceutical agent for at least 1 hour, at least 4 hours, at least 8 hours, at least 16 hours, or at least 24 hours.
1.34 Any of the preceding Compositions, wherein the drug ion exchange particle provides sustained release of the active pharmaceutical agent for up to 24 hours.
1.35 Any of the preceding Compositions, wherein the coated drug ion exchange particle provides sustained release of the active pharmaceutical agent for 8-24 hours.
1.36 Any of the preceding Compositions, wherein the majority of the Coated Granules have a particle size of about 150-250 microns.
1.37 Any of the preceding Compositions, wherein the active pharmaceutical agent comprises naltrexone or a pharmaceutically acceptable salt thereof.
1.38 Any of the preceding Compositions, wherein the Composition is in the form of a liquid suspension, and the Composition provides a pharmacokinetic profile in healthy subjects, having one or more of the following characteristics a-e:
   a) Tₘₐₓ of about 5 to about 7 hours, for example about 5 hours, about 6 hours or about 7 hours;
   b) T_{1/2} of about 5 to about 12 hours, for example about 5 to about 11 hours; about 6 hours, about 7 hours, about 8 hours, about 9 hours, about 10 hours or about 11 hours;
   c) Cₘₐₓ of about 1.5 to about 4 hours, for example about 1.5 hours, about 2 hours about 2.5 hours, about 3 hours, about 3.5 hours or about 4 hours;
   d) AUCₜ of about 26 to about 32 hr·ng/mL, for example about 26, about 27, about 28, about 29, about 30, about 31 or about 32 hr·ng/mL; and
   e) AUC_{∞} of about 28 to about 35 hr·ng/mL, for example about 28, about 29, about 30, about 31, about 32, about 33, about 34 of about 35 hr·ng/mL.
1.39 Composition 1.38, wherein the extended release suspension has a pharmacokinetic profile having two or more of the characteristics a-e, or three or more of the characteristics a-e, or four or more of the characteristics a-e, or all five of the characteristics a-e.
1.40 Composition 1.38 or 1.39, wherein the extended release suspension has a pharmacokinetic profile is substantially as shown for Formulation 1 or Formulation 2 in FIG. 4.
1.41 Any of the preceding Compositions 1.38 et seq., wherein the Mean Plasma Naltrexone Concentration (ng/ml) over the period 2.5 hours post administration to 4 hours post administration increases by least 4%; or from about 4% to about 20%; or from about 4% to about 15%; or from about 4% to about 12%, or from about 5% to about 12%, or by about 4%, about 5%, about 6%, about 7%, about 8%, about 9%, about 10%, about 11% or about 12%.
1.42 Any of the preceding Compositions 1.38 et seq., wherein the Mean Plasma Naltrexone Concentration (ng/ml) over the period 4 hours post administration to 6 hours post administration increases by least about 80%; or from about 80% to about 120%; or from about 80% to about 110%; or from about 85% to about 105%, or about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104% or about 105%.
1.43 Any of the preceding Compositions 1.38 et seq., wherein the Mean Plasma Naltrexone Concentration (ng/ml) over the period 6 hours post administration to 8 hours post administration decreases by no more than about 40%; or from about 15% to about 40%; or from about 20% to about 35%; or about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%.
1.44 Any of the preceding Compositions 1.38 et seq., wherein the ratio of the Mean Plasma Naltrexone Concentration (Cₜ; ng/ml) to the Maximum Plasma Naltrexone Concentration (Cₘₐₓ; ng/ml) three hours after administration is from about 0.4 to about 0.6; for example, from about 0.4 to about 0.55, for example from about 0.45 to about 0.5.
1.45 Any of the preceding Compositions 1.38 et seq., wherein the ratio of the Mean Plasma Naltrexone Concentration (Cₜ; ng/ml) to the Maximum Plasma Naltrexone Concentration (Cₘₐₓ; ng/ml) six hours after administration is from about 0.8 to about 1.2; for example, from about 0.9 to about 1.1, for example from about 0.95 to about 1.05.
1.46 Any of the preceding Compositions 1.38 et seq., wherein the ratio of the Mean Plasma Naltrexone Concentration (Cₜ; ng/ml) to the Maximum Plasma Naltrexone Concentration (Cₘₐₓ; ng/ml) eight hours after administration is from about 0.5 to about 0.9; for example, from about 0.6 to about 0.8, for example from about 0.65 to about 0.8.
1.47 Any of the preceding Compositions 1.38 et seq., wherein the ratio of the Mean Plasma Naltrexone Concentration (Cₜ; ng/ml) to the Maximum Plasma Naltrexone Concentration (Cₘₐₓ; ng/ml) ten hours after administration is from about 0.4 to about 0.8; for example, from about 0.5 to about 0.7.
1.48 Any of the preceding Compositions 1.38 et seq., wherein the ratio of the Mean Plasma Naltrexone Concentration (Cₜ; ng/ml) to the Maximum Plasma Naltrexone Concentration (Cₘₐₓ; ng/ml) twelve hours after administration is from about 0.3 to about 0.7; for example, from about 0.4 to about 0.6.
1.49 Any of the preceding Compositions 1.38 et seq., wherein the ratio of the Mean Plasma Naltrexone Concentration (Cₜ; ng/ml) to the Maximum Plasma Naltrexone Concentration (Cₘₐₓ; ng/ml) is one or more of a-e below:
   a) three hours after administration, from about 0.4 to about 0.6; for example, from about 0.4 to about 0.55, for example from about 0.45 to about 0.5;
   b) six hours after administration, from about 0.8 to about 1.2; for example, from about 0.9 to about 1.1, for example from about 0.95 to about 1.05;
   c) eight hours after administration, from about 0.5 to about 0.9; for example, from about 0.6 to about 0.8, for example from about 0.65 to about 0.8;
   d) ten hours after administration, from about 0.4 to about 0.8; for example, from about 0.5 to about 0.7; and
   e) twelve hours after administration, from about 0.3 to about 0.7; for example, from about 0.4 to about 0.6.
1.50 Composition 1.49, wherein the ratio of the Mean Plasma Naltrexone Concentration (Cₜ; ng/ml) to the Maximum Plasma Naltrexone Concentration (Cₘₐₓ; ng/ml) is
   a) three hours after administration, from about 0.4 to about 0.6; for example, from about 0.4 to about 0.55, for example from about 0.45 to about 0.5; and
   b) six hours after administration, from about 0.8 to about 1.2; for example, from about 0.9 to about 1.1, for example from about 0.95 to about 1.05.
1.51 Composition 1.49, wherein the ratio of the Mean Plasma Naltrexone Concentration (Cₜ; ng/ml) to the Maximum Plasma Naltrexone Concentration (Cₘₐₓ; ng/ml) is
   b) six hours after administration, from about 0.8 to about 1.2; for example, from about 0.9 to about 1.1, for example from about 0.95 to about 1.05; and
   c) eight hours after administration, from about 0.5 to about 0.9; for example, from about 0.6 to about 0.8, for example from about 0.65 to about 0.8.
1.52 Composition 1.49, wherein the ratio of the Mean Plasma Naltrexone Concentration (Cₜ; ng/ml) to the Maximum Plasma Naltrexone Concentration (Cₘₐₓ; ng/ml) is
   c) eight hours after administration, from about 0.5 to about 0.9; for example, from about 0.6 to about 0.8, for example from about 0.65 to about 0.8; and
   d) ten hours after administration, from about 0.4 to about 0.8; for example, from about 0.5 to about 0.7.
1.53 Composition 1.49, wherein the ratio of the Mean Plasma Naltrexone Concentration (Cₜ; ng/ml) to the Maximum Plasma Naltrexone Concentration (Cₘₐₓ; ng/ml) is
   a) three hours after administration, from about 0.4 to about 0.6; for example, from about 0.4 to about 0.55, for example from about 0.45 to about 0.5; and
   b) six hours after administration, from about 0.8 to about 1.2; for example, from about 0.9 to about 1.1, for example from about 0.95 to about 1.05; and
   c) eight hours after administration, from about 0.5 to about 0.9; for example, from about 0.6 to about 0.8, for example from about 0.65 to about 0.8; and
   d) ten hours after administration, from about 0.4 to about 0.8; for example, from about 0.5 to about 0.7
1.54 Any of the preceding Compositions, wherein no more than 25% of the active pharmaceutical agent is released within one hour and wherein no more than 60% of the active pharmaceutical agent is released within two hours.
1.55 Any of the preceding Compositions, wherein no more than 30% of the active pharmaceutical agent is released within one hour and wherein no more than 60% of the active pharmaceutical agent is released within two hours.

In a third aspect, the present disclosure provides a method [Method 1] for preparing a coated drug-ion exchange resin particle (or granule), comprising:
(a) contacting an active drug substance and an ion exchange resin in a solvent to form a drug-ion exchange resin complex (or resinous core);
(b) optionally isolating and drying the drug-ion exchange resin complex;
(c) granulating the drug-ion exchange resin complex to form a drug-ion exchange resin complex granule;
(d) optionally drying the product of step (c), for example to 15-25% or 15-20% moisture content, and optionally milling the product, for example using a co-mill fitted with about 400-micron mesh screen; or optionally screening the product to a predetermined size, for example by passing the product through a mesh screen, for example a 20 mesh screen;
(e) optionally further drying the product of step (d), for example to <7% moisture content;
(f) optionally milling the product of step (e), for example with a co-mill fitted with 813-micron mesh screen; and
(g) coating the product of the preceding steps with a coating composition comprising:
   a cellulose acetate-containing polymer, for example cellulose acetate butyrate; optionally, an enteric polymer;
   optionally, a plasticizer, e.g., polyethylene glycol, e.g. polyethylene glycol 3550; and a solvent for example water, acetone, or water and acetone;
   for example wherein the coating is applied to a weight gain of 15-20% w/w;
   to form the coated drug-ion exchange resin particle (or granule).

The disclosure further provides the following embodiments of Method 1:
1.1 Method 1, wherein the solvent in step (a) comprises water.
1.2 Method 1 and 1.1, wherein step (b) is performed, and the drug-ion exchange resin complex is isolated by filtration and dried, for example using a fluid bed processor.
1.3 Any of the preceding Methods, wherein step (b) is performed, and the drug-ion exchange resin complex is dried until the moisture content is about 15%.
1.4 Any of the preceding Methods, wherein the granulation step (c) comprises:
   i. contacting the product of step (b) with a composition comprising a solvent (e.g. water), optionally a chelating agent (e.g. EDTA) and optionally a granulating agent (e.g. polyethylene glycol, e.g. polyethylene glycol 3550); and
   ii. granulating the product of step (i) to form an uncoated drug-ion exchange resin complex granule.
1.5 Method 1.4, wherein step (i) is performed by spraying the product of step (b) with the composition.
1.6 Method 1.4 or 1.5, wherein step ii is performed using a high shear granulator.
1.7 Any of the preceding Methods, wherein step (c) further comprises milling the drug-ion exchange resin complex granules and separating them by size (for example by sieving; e.g. with a 400 micron mesh screen).
1.8 Any of the preceding Methods, wherein the coating composition is sprayed onto the drug-ion exchange resin complex granules to a weight gain of 15% w/w to 20% w/w.
1.9 Any of the preceding Methods, wherein step (d) is performed.
1.10 Any of the preceding Methods, wherein step (d) is performed, and the product of step (c) is dried to 15-25% moisture content.
1.11 Method 1.9, wherein the product having 15-25% moisture content is passed through a 20 mesh screen.
1.12 Any of the preceding Methods, wherein step (e) is performed, wherein the product of step (d) is dried to <7% moisture content.
1.13 Any of the preceding Methods, wherein step (f) is performed.
1.14 Method 1.12, wherein the milling in step (f) is performed with a co-mill fitted with an 813-micron mesh screen.
1.15 Any of the preceding Methods, wherein in step (g) the coating composition is sprayed onto the uncoated drug-ion exchange resin complex granules.
1.16 Any of the preceding Methods, wherein in step (g) the coating composition comprises one or more solvents.
1.17 Any of the preceding Methods, wherein the coating composition comprises one or more solvents selected from water, acetone, ethanol, isopropyl alcohol and methanol; for example wherein the one or more solvents selected from water, acetone, and a combination thereof.
1.18 Any of the preceding Methods, wherein the coating composition comprises a plasticizer, e.g., polyethylene glycol.
1.19 Any of the preceding Methods, wherein step (g) comprises contacting the drug-ion exchange resin complex granules produced by the preceding steps with a coating composition comprising a cellulose acetate butyrate and polyethylene glycol in a solvent, e.g. a water-acetone mixture.
1.20 Any of the preceding Methods, wherein the ion exchange resin comprises a polystyrene polymer.
1.21 Any of the preceding Methods, wherein the ion exchange resin comprises a co-polymer of polystyrene and divinylbenzene.
1.22 Any of the preceding Methods, wherein the ion exchange resin comprises a matrix of sulfonated polymers composed of polystyrene cross-linked with about 8% of divinylbenzene 1.23 Any of the preceding Methods, wherein the ion exchange resin has an ion-exchange capacity of about 4.5 to 5.5 meq/g of dry resin.
1.24 Any of the preceding Methods, wherein the resinous core comprises a particle size of about 45 microns to 150 microns.
1.25 Any of the preceding Methods, wherein the resinous core comprises particles which are spherical or irregularly shaped.
1.26 Any of the preceding Methods, wherein the ion exchange resin comprises Amberlite IRP-69.
1.27 Any of the preceding Methods, wherein the ion exchange resin and the one or more active pharmaceutical agents are present in a w/w ratio of about 5:1 to 1:5, e.g., about 3:1 to 1:3, e.g., about 2:1 to 1:2, e.g., about 2:1.
1.28 Any of the preceding Methods, wherein the cellulose acetate-containing polymer is selected from cellulose acetate, cellulose diacetate, cellulose triacetate, cellulose acetate butyrate, cellulose acetate phthalate, cellulose acetate propionate, and combinations thereof.
1.29 Any of the preceding methods, wherein the cellulose acetate polymer is selected from cellulose acetate, cellulose acetate butyrate, and combinations thereof.
1.30 Any of the preceding Methods, wherein the cellulose acetate polymer comprises cellulose acetate.
1.31 Any of the preceding Methods, wherein the cellulose acetate polymer is cellulose acetate.
1.32 Any of the preceding Methods, wherein the cellulose acetate polymer comprises cellulose acetate butyrate.
1.33 Any of the preceding Methods, wherein the cellulose acetate polymer is cellulose acetate butyrate.
1.34 Any of the preceding Methods, wherein the coating composition in step (g) comprises an enteric polymer selected from a phthalate-containing polymer; cellulose acetate trimellitate; polymethacrylates; hydroxypropylmethylcellulose acetate succinate (HPMC AS; Aqoat®); sodium alginate; alginic acid; shellac and combinations thereof.
1.35 Any of the preceding Methods, wherein the enteric polymer is or comprises a phthalate-containing polymer comprising monomers of dimethyl phthalate, diethyl phthalate, diallyl phthalate, di-n-propyl phthalate, di-n-butyl phthalate, diisobutyl phthalate, butyl cyclohexyl phthalate, di-n-pentyl phthalate, dicylcohexyl phthalate, butyl benzyl phthalate, di-n-hexyl phthalate, diisohexyl phthalate, diisoheptyl phthalate, butyl decyl phthalate, dibutoxy ethyl phthalate, di(2-ethylhexyl) phthalate, di(n-octyl) phthalate, diisooctyl phthalate, n-octyl n-decyl phthalate, diisononyl phthalate, di(2-propylheptyl) phthalate, diisodecyl phthalate, diundecyl phthalate, diisoundecyl phthalate, ditridecyl phthalate, diisotridecyl phthalate, polyethylene terephthalate, cellulose phthalate, cellulose acetate phthalate (CAP), hydroxypropylmethylcellulose phthalate (HPMCP), polyvinyl acetate phthalate (PVAP) and combinations thereof.
1.36 Any of the preceding Methods, wherein the enteric polymer comprises a phthalate-containing polymer selected from cellulose acetate phthalate (CAP), hypromellose phthalate (hydroxypropylmethylcellulose phthalate; HPMCP), and polyvinyl acetate phthalate (PVAP).
1.37 Any of the preceding Methods, wherein the enteric polymer is a phthalate-containing polymer selected from cellulose acetate phthalate (CAP), hypromellose phthalate (hydroxypropylmethylcellulose phthalate; HPMCP), and polyvinyl acetate phthalate (PVAP).
1.38 Any of the preceding Methods, wherein the enteric polymer comprises cellulose acetate trimellitate.
1.39 Any of the preceding Methods, wherein the coated drug ion exchange particle further comprises an anti-tacking agent; for example and not limited to an anti-tacking agent such as talc, glycerol monostearate, magnesium stearate or kaolin.
1.40 Any of the preceding Methods, wherein the enteric polymer comprises a polymethacrylate.
1.41 Any of the preceding Methods, wherein the enteric polymer is a polymethacrylate. 1.42 Any of the preceding Methods, wherein the enteric polymer comprises a polymethacrylate selected from Eudragit® L series, Eudragit® S series, Eudragit® FS, Kollicoat® MAE 100P, Acryl-EZE® 93A, and Acryl-EZE® MP.
1.43 Any of the preceding Methods, wherein the enteric polymer is a polymethacrylate selected from Eudragit® L series, Eudragit® S series, Eudragit® FS, Kollicoat® MAE 100P, Acryl-EZE® 93A, or Acryl-EZE® MP.
1.44 Any of the preceding Methods, wherein the enteric polymer comprises hydroxypropylmethylcellulose acetate succinate (HPMC AS; Aqoat®).
1.45 Any of the preceding Methods, wherein the enteric polymer is hydroxypropylmethylcellulose acetate succinate (HPMC AS; Aqoat®). 1.46 Any of the preceding Methods, wherein the enteric polymer comprises sodium alginate or alginic acid.
1.47 Any of the preceding Methods, wherein the enteric polymer is sodium alginate or alginic acid.
1.48 Any of the preceding Methods, wherein the enteric polymer comprises shellac.
1.49 Any of the preceding Methods, wherein the enteric polymer is shellac.
1.50 Any of the preceding Methods, wherein the coating composition comprises a plasticizer.
1.51 Any of the preceding Methods, wherein the plasticizer in the coating composition is selected from benzophenone, butyl phthalyl butyl glycollate, camphor, alpha-cresyl-p-toluene sulfonate, cyclohexyl-p-toluene sulfonamide, diamyl phthalate, dibutyl phthalate, dibutyl sebacate, dibutyl succinate, dibutyl tartrate, diethoxyethyl adipate, diethoxyethyl phthalate, diethyl adipate, diethylene glycol dipropionate, diethyl phthalate, diethyl sebacate, diethyl succinate, diethyl tartrate, dimethoxyethyl adipate, dimethoxyethyl phthalate, dimethyl phthalate, dipropyl phthalate, ethyl benzoyl benzoate, ethylene glycol diacetate, ethylene glycol dibutyrate, ethylene glycol dipropionate, ethyl phthalyl ethyl glycolate, methyl benzoyl benzoate, methyl phthalyl ethyl glycolate, o- or p-toluene ethyl sulfonamide, triacetin, tributyl citrate, tributyl phosphate, tributyrin, tricresyl phosphate, triethylene glycol diacetate, triethylene glycol dibutyrate, triethylene glycol diproprionate, triphenyl phosphate, tripropionin, trimellitates (e.g., tri-(2-ethylhexyl) trimellitate, tri-(isononyl) trimellitate, tri-(isodecyl) trimellitate, tri-(isotridecyl) trimellitate), adipates (e.g., bis(2-ethylhexyl) adipate), sebacates (e.g., dibutyl sebacate, di-(2-ethylhexyl) sebacate), glycerol triacetate, alkyl citrates (triethyl citrate, acetyl triethyl citrate, tributyl citrate, acetyl trybutyl citrate, tri (2-ethylhexyl) citrate, acetyl trioctyl citrate, trihexyl citrate, butryyl trihexyl citrate), vegetable oils, epoxidized soybean oil, epoxidized linoleic oil, azelates, dibenzoates, terephthalates, 1,2-cyclohexane dicarboxylic acid diisononyl ester, alkyl sulphonic acid phenyl ester, organophosphates (e.g., tricresyl (methyl phenyl) phosphate, 2-ethylhexyldiphenyl phosphate), glycols (e.g., propylene glycol, polyethylene glycol, triethylene glycol di-2hexanoate) triacetin, triethyl citrate, dibutyl sebacate, vegetable oil, lipids and combinations thereof.
1.52 Any of the preceding Methods, wherein the plasticizer in the coating composition is selected from propylene glycol, polyethylene glycol, triacetin, triethyl citrate, acetyl triethyl citrate, dibutyl sebacate, acetyl tributyl citrate, tributyl citrate, Soluphor P, vegetable oil, lipids, and combinations thereof.
1.53 Any of the preceding Methods, wherein the plasticizer is dispersed throughout the coating.
1.54 Any of the preceding Methods, wherein the plasticizer comprises polyethylene glycol; for example polyethylene glycol 3350.
1.55 Any of the preceding Methods, wherein the plasticizer consists of polyethylene glycol; for example polyethylene glycol 3350.
1.56 Any of the preceding Methods, wherein the plasticizer is dispersed throughout the coating.
1.57 Any of the preceding Methods, wherein the plasticizer comprises about 2.5 to about 25% w/w, or about 5 to about 15% w/w, or about 8 to about 12% w/w, or about 10% w/w of the solids in the coating of solids in the coating.
1.58 Any of the preceding Methods 1.4 et seq., wherein the composition in step (c) (i) comprises a solvent (e.g. water), optionally a chelating agent (e.g. EDTA) and a granulating agent.
1.59 Method 1.58, wherein the granulating agent is selected from benzophenone, butyl phthalyl butyl glycollate, camphor, alpha-cresyl-p-toluene sulfonate, cyclohexyl-p-toluene sulfonamide, diamyl phthalate, dibutyl phthalate, dibutyl sebacate, dibutyl succinate, dibutyl tartrate, diethoxyethyl adipate, diethoxyethyl phthalate, diethyl adipate, diethylene glycol dipropionate, diethyl phthalate, diethyl sebacate, diethyl succinate, diethyl tartrate, dimethoxyethyl adipate, dimethoxyethyl phthalate, dimethyl phthalate, dipropyl phthalate, ethyl benzoyl benzoate, ethylene glycol diacetate, ethylene glycol dibutyrate, ethylene glycol dipropionate, ethyl phthalyl ethyl glycolate, methyl benzoyl benzoate, methyl phthalyl ethyl glycolate, o- or p-toluene ethyl sulfonamide, triacetin, tributyl citrate, tributyl phosphate, tributyrin, tricresyl phosphate, triethylene glycol diacetate, triethylene glycol dibutyrate, triethylene glycol diproprionate, triphenyl phosphate, tripropionin, trimellitates (e.g., tri-(2-ethylhexyl) trimellitate, tri-(isononyl) trimellitate, tri-(isodecyl) trimellitate, tri-(isotridecyl) trimellitate), adipates (e.g., bis(2-ethylhexyl) adipate), sebacates (e.g., dibutyl sebacate, di-(2-ethylhexyl) sebacate), glycerol triacetate, alkyl citrates (triethyl citrate, acetyl triethyl citrate, tributyl citrate, acetyl trybutyl citrate, tri (2-ethylhexyl) citrate, acetyl trioctyl citrate, trihexyl citrate, butyryl trihexyl citrate), vegetable oils, epoxidized soybean oil, epoxidized linoleic oil, azelates, dibenzoates, terephthalates, 1,2-cyclohexane dicarboxylic acid diisononyl ester, alkyl sulphonic acid phenyl ester, organophosphates (e.g., tricresyl (methyl phenyl) phosphate, 2-ethylhexyldiphenyl phosphate), glycols (e.g., propylene glycol, polyethylene glycol, triethylene glycol di-2hexanoate) triacetin, triethyl citrate, dibutyl sebacate, vegetable oil, lipids and combinations thereof.
1.60 Method 1.59, wherein the granulating agent comprises polyethylene glycol; for example polyethylene glycol 3350.
1.61 Method 1.59, wherein the granulating agent consists of polyethylene glycol; for example polyethylene glycol 3350.
1.62 Any of the preceding Methods, wherein the coated drug-ion exchange resin granule further comprises a plurality of active pharmaceutical agents (e.g., 2-10 active pharmaceutical agents).
1.63 Any of the preceding Methods, wherein the resinous core is complexed with one or more active pharmaceutical agents (e.g., one or more small molecule drugs, e.g., up to 10 small molecule drugs).
1.64 Any of the preceding Methods, wherein the active pharmaceutical agent is a free base compound.
1.65 Any of the preceding Methods, wherein the active pharmaceutical agent is a compound which may be protonated at one or more locations on the molecule.
1.66 Any of the preceding Methods, wherein the active pharmaceutical agent comprises naltrexone or a pharmaceutically acceptable salt thereof.
1.67 Any of the preceding Methods, wherein the active pharmaceutical agent is present in an amount of about 1% to about 99% by weight based on the total weight of the coated drug ion exchange resin complex granule, from about 5% to about 70% by weight, from about 10% to about 30% by weight, from about 20% to about 30% by weight, from about 20% to about 25% by weight, or about 22-24% by weigh based on the total weight of the coated drug-ion exchange resin complex granule; or about 22%, about 23% or about 24% by weigh based on the total weight of the coated drug-ion exchange resin granule.
1.68 Any of the preceding Methods, further comprising the step of:
   h) combining the coated drug-ion exchange resin particle (or granule) produced by step (g) and a suspension base composition to form a coated drug-ion exchange resin extended-release suspension.
1.69 Method 1.68, wherein the suspension base composition is in the form of a liquid suspension, and comprises one or more solvents, and one or more ingredients selected from sugars, antioxidants, chelating agents, buffering agents, thickeners, stabilizers, preservatives, surfactants, flavorings and coloring agents.
1.70 Method 1.68 or 1.69, wherein the solvent in the drug-ion exchange resin extended-release suspension comprises water.
1.71 Any of the preceding Methods 1.68 et seq., wherein the drug-ion exchange resin extended-release suspension comprises the Coated Granules in an amount of from about 0.01 g to about 0.03 g of Coated Granules per ml of suspension.
1.72 Any of the preceding Methods 1.68 et seq., wherein the Coated Granules are present in the drug-ion exchange resin extended-release suspension in an amount of about 0.01% w/v to about 10% w/v, based on the total weight of the composition; or about 0.01% w/v to about 10% w/v, based on the total weight of the composition; or about 0.1% w/v to about 5% w/v, based on the total weight of the composition; or about 0.1% w/v to about 5% w/v, based on the total weight of the composition; or about 1% w/v to about 5% w/v, based on the total weight of the composition; or about 1% w/v to about 5% w/v, based on the total weight of the composition; or about 2% w/v to about 3% w/v, based on the total weight of the composition; or about 2% w/v to about 3% w/v, based on the total weight of the composition.
1.73 Any of the preceding Methods 1.68 et seq., wherein the drug-ion exchange resin extended-release suspension is in the form of a suspension, for example a liquid suspension, and the active pharmaceutical agent is present in an amount from about 0.1 mg/mL to about 100 mg/mL, e.g., from about 1 mg/mL to about 25 mg/mL, e.g., from about 3 mg/mL to about 8 mg/mL, e.g., about 5 mg/mL.
1.74 Any of the preceding Methods 1.68 et seq., wherein the drug-ion exchange resin extended-release suspension is in the form of a suspension, for example a liquid suspension, and the active pharmaceutical agent is present in an amount from about 0.1% w/v to about 2% w/v, e.g., from about 0.1% to about 1% w/v; e.g., from about 0.2% w/v to about 0.8% w/v, e.g., from about 0.4% w/v to about 0.6% w/v, e.g. about 0.5% w/v, e.g. 0.49%-0.51% w/v.
1.75 Any of the preceding Methods 1.68 et seq., wherein the drug-ion exchange resin extended-release suspension comprises a plurality of said Coated Granules.
1.76 Any of the preceding Methods 1.68 et seq., wherein the Coated Granules are present in an amount of from 0.5% to about 5% w/v; for example from about 1.0% to about 3.0% w/v; for example from 2.0% to about 2.5% w/v, for example about 2.3% w/v.
1.77 Any of the preceding Methods 1.68 et seq., wherein the drug-ion exchange resin extended-release suspension comprises:
   a chelating agent, for example EDTA, for example wherein the EDTA is present in an amount of from about 0.05% to about 0.15% w/v; for example from about 0.08% to about 0.12% w/v; for example from about 0.09% to about 0.10% w/v; for example about 0.095% w/v;
   a buffering agent, for example citric acid, for example wherein the citric acid is present in an amount of from about 0.1% to about 0.8% w/v; for example from about 0.2% to about 0.6% w/v; for example from about 0.3% to about 0.5% w/v; for example about 0.4% w/v;
   an antioxidant, for example propyl gallate, in an amount of from about 0.01% to about 0.03%, for example about 0.02%;
   a surfactant, for example Tween 80, in an amount of from about 0.01% to about 0.3% w/v; for example from about 0.05% to about 0.2% w/v; for example about 0.1% w/v;
   a solvent, for example water;
   a sugar, for example sucrose, in an amount of from about 5% to about 40% w/v;
   for example, from about 10% to about 30% w/v; for example, from about 15% to about 25% w/v; for example, about 20% w/v;
   a preservative, for example sodium benzoate, in an amount of from about 0.1% to about 0.5% w/v; for example from about 0.15% to about 0.3% w/v; for example about 0.2% w/v; and
   a thickener, for example starch, xanthan gum, or a combination of starch and xanthan gum, for example both starch and xanthan gum, for example wherein the starch is present in an amount of from about 0.1% to about 5% w/v; for example from about 1% to about 3% w/v; for example about 2% w/v; and the xanthan gum is present in an amount of from about 0.05% to about 0.5% w/v; for example from about 0.1% to about 0.2% w/v; for example about 0.15% w/v.
1.78 Any of the preceding Methods 1.68 et seq., wherein the drug-ion exchange resin extended-release suspension comprises:
   a Coated Granule according to any of Coated Granules 1 or 1.1-1.86;
   a sugar (e.g. sucrose);
   a chelating agent (e.g. EDTA);
   a buffering agent (e.g. citric acid, e.g. citric acid anhydrous);
   one or more thickeners (e.g. starch and/or Xanthan gum);
   a preservative (e.g. sodium benzoate);
   a surfactant (e.g. TWEEN 80); and
   an antioxidant (e.g. propyl gallate).
1.79 Any of the preceding Methods 1.68 et seq., wherein the drug-ion exchange resin extended-release suspension has the following composition:

| Ingredient | Quantity (g) | % w/v |
|---|---|---|
| Sugar (e.g. Sucrose) | 600-1000; e.g. 800 | 5-40; e.g. 10- |
| | | 30%; e.g. 15%- |
| | | 25%; e.g. 20 |
| Buffering agent (e.g. citric acid | 10-20; e.g. 14-18; e.g. 16 | 0.1-0.8; e.g. 0.2- |
| anhydrous) | | 0.6; e.g. 0.25-0.5; |
| | | e.g. 0.3-0.5; e.g. |
| | | 0.35-0.45; e.g. 0.4 |
| Chelating agent (e.g. EDTA) | 2-6; e.g. 3-5; e.g. 3.5-4.5; e.g., 3.8 | 0.05-0.15; e.g. |
| | | 0.075-0.125; e.g. |
| | | 0.08-0.12; e.g. |
| | | 0.09-0.10; e.g. |
| | | 0.095 |
| Thickener (e.g. Starch) | 70-100; e.g. 80-90; e.g. 84-85, e.g. | 0.1-5; e.g. 1-3; |
| | 84.39 | e.g. 1.75-2.5; e.g. |
| | | 2.0-2.25; e.g. 2.1- |
| | | 2.3; e.g. 2; e.g., |
| | | 2.1 |
| Preservative (e.g. Sodium | 4-12; e.g. 6-10; e.g. 8 | 0.1-0.5; e.g. 0.1- |
| benzoate) | | 0.3; e.g. 0.15- |
| | | 0.25; e.g.0.2 |
| Thickener (e.g. Xanthan gum) | 2-10; e.g. 4-8; e.g. 5-7; e.g. 6 | 0.05-0.5; e.g. 0.1- |
| | | 0.2; e.g. 0.15 |
| Surfactant (e.g. Tween 80) | 2-6; e.g. 3-5; e.g. 4 | 0.01-0.3; e.g. |
| | | 0.05-0.2; e.g. 0.1 |
| Anti-oxidant (e.g. propyl | 0.4-1.2; e.g. 0.6-1.0; e.g. 0.8 | 0.01-0.03; e.g. |
| gallate) | | 0.02 |
| Coated Granules | 60-110; e.g. 70-100; e.g., 80-90; e.g. | 0.5-5; e.g. 1.0- |
| | 85; e.g., 89 | 3.0; e.g. 2.0-2.5; |
| | | e.g. 2.3; e.g. about |
| | | 2.28 |
| Color agent (e.g. FD & C red | 0.1-1.3; e.g. 0.2 | 0.005 |
| 40) | | |
| Flavor (e.g. Cherry flavor) | 2-6; e.g. 4 | 0.1 |
| Purified water | QS to 4 L | QS to 4 L |

1.80 Any of the preceding Methods 1.68 et seq., wherein the coated drug-ion exchange resin extended-release suspension is a Composition according to any of Compositions 1 and 1.1-1.55 herein.
1.81 Any of the preceding Methods 1.68 et seq., wherein the coated drug-ion exchange resin extended-release suspension does not contain glycerin, or polyethylene glycol, other than the granulating agent and plasticizer in the coated granules.

As used herein, the terms “coated drug-ion exchange resin particle,” “coated drug-ion exchange resin granule” and “coated particles” and “Coated Granule” are intended to have the same meaning; i.e., the composition comprising:
a drug-ion exchange resin complex (or resinous core) comprising:
   an ion exchange resin;
   one or more active pharmaceutical agents;
   optionally a chelating agent;
   optionally a granulating agent; and
a coating comprising:
   a cellulose acetate-containing polymer;
   optionally, an enteric polymer; and
   optionally, a plasticizer
as described above.

The term “contacting” as used herein in the steps of the present Methods is intended to mean that the indicated components are placed in contact with each other, either directly or in a suitable solvent, such that the indicated reaction or complex formation occurs.

As used herein, the term “substantially” when used in connection with the pharmacokinetic profiles and the Figures herein is intended to mean that the values for AUCₜ, AUC_{∞} and Cₘₐₓ are within +/−20% of the mean values for AUCₜ, AUC_{∞} and Cₘₐₓ shown in the profile.

In various embodiments, the coated particles or granules of the present disclosure are irregular in shape. However, a proportion of the particles or granules may be spherical or polyhedral in shape. Thus, as used herein, the term “particle size” is generally used to refer to one or more of the following: (a) the diameter of a spherical particle, (b) the length of the longest axis of the particle, (c) the length of the shortest axis of the particle, (d) any measure between the length of the long axis and the length of the short axis, including the mean between the two. In various embodiments, particle size is determined according to the length of the longest axis of the particle. Diameter is generally expressed in micrometers (μm or micron). Diameter may be determined by any appropriate means known in the art, e.g., via optical measurement.

The coated particles granules of the present disclosure comprise a resinous core surrounded by a polymer coating. A drug resin complex or drug-ion exchange resin complex may be formed by mixing a drug, e.g. naltrexone, or a salt thereof, for example naltrexone hydrochloride, and a resin, e.g. an ion-exchange resin (e.g., a polystyrene sulfonate resin) to facilitate protonation of the drug and subsequent bonding (e.g., ionic bonding) between the resinous polymer and the drug. Without being bound by theory, it is believed the drug bonds to the resinous polymer within the interior of the core as well as on the surface of the core. It is acknowledged that various drugs will exhibit variable affinities for bonding with the resinous polymer. This reaction may be carried out in an excess of drug until the drug no longer binds to the resinous polymer.

Binding of the selected drug or combination of drugs to the ion exchange resin can be accomplished using methods known in the art. Several different reaction types may be used for binding of a basic drug to the resinous polymer of the present disclosure. For example, the resinous polymer (e.g., in a sodium salt form) may be reacted with a drug in salt or free base form. Alternatively, the resinous polymer (e.g., in a form capable of being deprotonated) may be reacted with the drug in salt or free base form. These reactions may have cationic by-products and these by-products, by competing with the cationic drug for binding sites on the resinous polymer, can reduce the amount of drug bound at equilibrium.

The drug resin complex thus formed is collected by filtration and washed with appropriate solvents to remove any unbound drug or by-products. The complexes can be air-dried in trays, in a fluid bed dryer, or other suitable dryer, at room temperature or at elevated temperature.

Batch equilibration may be used for preparing the complexes by loading a drug into finely divided resin powders. It will be acknowledged that the capacity of a resinous polymer to bind to any particular drug will be influenced by such factors as the inherent selectivity of the resin for the drug, the concentration of the drug in the loading solution, and the concentration of competing ions also present in the loading solution. The rate of binding will be affected by the activity of the drug and its molecular dimensions as well as the extent to which the polymer phase is swollen during loading.

It is usually desirable to load as much drug as possible onto the resin. Complete transfer of the drug from the loading solution is not likely in a single equilibrium stage. Accordingly, more than one equilibration may be required in order to achieve the desired loading onto the ion exchange resin. The use of two or more loading stages, separating the resin from the liquid phase between stages, is a means of achieving maximum loading of the drug onto the resin.

Without being bound by theory, drugs with higher charge densities will generally exhibit higher loading capacity onto the resin. Similarly, drugs with relatively lower molecular weights will load more efficiently than drugs with relatively higher molecular weights. Higher drug concentrations in the loading solution, with a minimum of competing ions, will also favor higher adsorption capacity onto the resin.

In various embodiments, the amount of drug that can be loaded onto a resin ranges from about 1% to about 75% by weight of the drug-ion exchange resin complex. In some aspects of Embodiments 1 and 2, drug loads of about 10% to about 40% by weight, e.g., about 15% to about 30% by weight, of the drug-ion exchange resin complex may be achieved.

In some embodiments, the drug-ion exchange resin complex is granulated, for example by wet granulation. Typically, the drug-ion exchange resin complex is contacted with a composition comprising a solvent (e.g. water), optionally a chelating agent (e.g. EDTA) and a granulating agent (e.g. polyethylene glycol, e.g. polyethylene glycol 3550), to form a drug-ion exchange resin complex granule, for example by spraying the composition on to the drug-ion exchange resin complex, and granulating the mixture, for example by using a high shear granulator. Suitable granulating agents are water soluble and water insoluble. Examples of suitable granulating agents include, e.g., dibutyl sebacate, propylene glycol, polyethylene glycol, polyvinyl alcohol, triethyl citrate, acetyl triethyl citrate, acetyl tributyl citrate, tributyl citrate, triacetin, and Soluphor P, and mixtures thereof. In some embodiments, the granulating agent comprises or consists of polyethylene glycol. Any molecular weight polyethylene glycol can be used. In some embodiments, the granulating agent comprises or consists of polyethylene glycol 3550.

The polymer coating, in various embodiments, comprises a combination of a cellulose acetate-containing polymer, alone or in combination with one or more enteric polymers. As used herein, the term “cellulose acetate-containing polymer” is intended to mean a polymer comprising at least one mono-, di- or tri-acetylated cellulose (i.e., β (1->4) linked glucose) monomer. In various embodiments, the cellulose acetate-containing polymer may be selected from cellulose acetate, cellulose diacetate, cellulose triacetate, cellulose acetate butyrate, cellulose acetate propionate, and combinations thereof. In some preferred embodiments, the cellulose acetate polymer is selected from cellulose acetate and cellulose acetate butyrate. In some further preferred embodiments, the cellulose acetate polymer comprises or consists of cellulose acetate butyrate.

In some embodiments, the coated drug-ion exchange resin complex particles and/or granules further comprise an enteric polymer, which can be selected from a phthalate-containing polymer; cellulose acetate trimellitate; polymethacrylates; hydroxypropylmethylcellulose acetate succinate (HPMC AS; Aqoat®); sodium alginate; alginic acid; shellac and combinations thereof.

In some embodiments, the enteric polymer is, or comprises, a phthalate-containing polymer; i.e., a polymer that comprises phthalate-containing monomers. In various embodiments, the enteric polymer is or comprises a phthalate-containing polymer comprising monomers of dimethyl phthalate, diethyl phthalate, diallyl phthalate, di-n-propyl phthalate, di-n-butyl phthalate, diisobutyl phthalate, butyl cyclohexyl phthalate, di-n-pentyl phthalate, dicylcohexyl phthalate, butyl benzyl phthalate, di-n-hexyl phthalate, diisohexyl phthalate, diisoheptyl phthalate, butyl decyl phthalate, dibutoxy ethyl phthalate, di(2-ethylhexyl) phthalate, di(n-octyl) phthalate, diisooctyl phthalate, n-octyl n-decyl phthalate, diisononyl phthalate, di(2-propylheptyl) phthalate, diisodecyl phthalate, diundecyl phthalate, diisoundecyl phthalate, ditridecyl phthalate, diisotridecyl phthalate, polyethylene terephthalate, cellulose phthalate, cellulose acetate phthalate (CAP), hydroxypropylmethylcellulose phthalate (HPMCP), polyvinyl acetate phthalate (PVAP) or combinations thereof.

In some preferred embodiments, the phthalate-containing polymer is selected from cellulose acetate phthalate (CAP), hypromellose phthalate (hydroxypropylmethylcellulose phthalate; HPMCP), and polyvinyl acetate phthalate (PVAP).

In some embodiments, the enteric polymer can comprise cellulose acetate trimellitate. In some embodiments, the enteric polymer can comprise a polymethacrylate, for example a polymethacrylate selected from a methacrylic acid-methyl methacrylate copolymer (1:1) such as Eudragit® L series, a methacrylic acid-methyl methacrylate copolymer (1:2) such as Eudragit® s series, a poly(methyl acrylate-co-methyl methacrylate-co-methacrylic acid) such as Eudragit® FS, or a methacrylic acid-ethyl acrylate copolymer (1:1) such as Kollicoat® MAE 100P. In some aspects of Embodiments 1 and 2, the enteric polymer can comprise an acrylic enteric coating system, for example Acryl-EZE® 93A, and Acryl-EZER MP.

In some further embodiments, the enteric polymer can comprise hydroxypropylmethylcellulose acetate succinate (HPMC AS; Aqoat®); sodium alginate; alginic acid; shellac and combinations thereof.

In some embodiments, the polymer coating comprises about 1% to about 30%, by weight, of the uncoated drug-ion exchange resin complex granule (i.e., the drug bound to the resinous polymer in particle form), e.g., about 3% to about 25% by weight, about 10% to about 20% by weight, or about 15% by weight of the uncoated drug resin complex.

In some embodiments, the barrier coating further contains a plasticizer, e.g., one or more of the plasticizers disclosed herein, which can facilitate uniform coating of the drug-ion exchange resin complex and/or enhances the tensile strength of the barrier coating layer.

Generally, a plasticizer is used in the percent range, or a mixture of plasticizers combine to total, about 2 to about 50% by weight of the polymer coating, more preferably about 2.5% to about 25% by weight of the coating layer on the coated drug resin complex. Preferably a plasticizer in range of about 5% to about 15% by weight of the coating layer provides the most desirable properties.

Suitable plasticizers are water soluble and water insoluble. Examples of suitable plasticizers include, e.g., dibutyl sebacate, propylene glycol, polyethylene glycol, polyvinyl alcohol, triethyl citrate, acetyl triethyl citrate, acetyl tributyl citrate, tributyl citrate, triacetin, and Soluphor P, and mixtures thereof. In some preferred embodiments, the granulating agent in the resinous core and the plasticizer in the coating each independently comprises or consists of polyethylene glycol. Any molecular weight polyethylene glycol can be used. In some embodiments, the granulating agent comprises or consists of polyethylene glycol 3550.

In some embodiments, the release rate of the polymer coatings of the present disclosure for at least 1 hour, at least 4 hours, at least 8 hours, at least 16 hours, at least 24 hours, In some embodiments, the disclosed polymer coatings have a release rate over a period of about 8 to 24 hours, and preferably 12 to 24 hours.

The coated drug-ion exchange resin granules of the present disclosure can readily be formulated with pharmaceutically acceptable excipients according to methods well known to those of skill in the art. In certain embodiments, these formulations contain a coated drug ion-exchange resin complex particles of the present disclosure. In some embodiments, mixtures of coated drug-ion exchange resin complex particles and uncoated drug-ion exchange resin complex particles may be present. These formulations may contain any suitable ratio of coated to uncoated particles.

It is further envisioned that the formulations of the present disclosure may contain more than one active pharmaceutical agent. For example, the formulation may contain more than one drug loaded into the resinous polymer to form a complex. As another example, the formulation may contain a first coated drug-ion exchange resin complex particle containing a first active pharmaceutical agent of the disclosure in combination with a second coated drug-ion exchange resin complex particle containing a second active pharmaceutical agent. In still another example, the formulation may contain a coated drug-ion exchange resin particle in combination with one or more active pharmaceutical agents which are not in a coated drug-ion exchange resin particle or granule.

The coated drug-ion exchange resin particles of the present disclosure are typically formulated for oral delivery.

The resin composition thus prepared may be stored for future use or promptly formulated with conventional pharmaceutically acceptable carriers to prepare finished ingestible compositions for oral delivery. The compositions according to the present disclosure may, for example, take the form of liquid preparations such as suspensions, or solid preparations such as capsules, sachets, tablets, chewable tablets, caplets, sublinguals, powders, wafers, strips, gels, including liquigels, etc. In one embodiment, a tablet of the disclosure is formulated as an orally disintegrating tablet (ODT). Such orally dissolving tablets may disintegrate in the mouth in less than about 60 seconds.

Compositions according to the present disclosure which comprise coated drug-ion exchange resin complex particles and granules may be formulated using conventional pharmaceutically acceptable carriers or excipients and known techniques. For example, the disclosed compositions may include conventional carriers or excipients include diluents (e.g., microcrystalline cellulose, lactose), thickeners (e.g., starch, xanthan gum), binders (e.g., cellulose derivatives and acrylic derivatives), lubricants (i.e., magnesium or calcium stearate, or vegetable oils, polyethylene glycols, talc, sodium lauryl sulfate, amorphous silica, polyoxy ethylene monostearate), thickeners, humectants, disintegrants (e.g., crospovidone), antioxidants (propyl gallate, ascorbic acid, sodium metabisulfite, butylated hydroxy anisole, butylated hydroxy toluene), colorants, flavorants, preservatives, sweeteners, buffers, as well as other components which will be readily apparent to one of ordinary skill in the art.

### Carriers

Suitable pharmaceutically acceptable carriers may include one or more of fatty acid esters, fatty acids and salts thereof, fatty alcohols (such as lauryl, myristyl stearyl, cetyl or cetostearyl alcohol), fatty amines, fatty amides, glycerides (e.g., mono-, di- and tri-glycerides), hydrogenated fats, glycolipids, steroids, natural and synthetic waxes, polyethylene glycol (PEG) or derivatives, and the like. Specific examples include, but are not limited to, hydrogenated vegetable oil, hydrogenated cottonseed oil, hydrogenated castor oil, stearic acid, cocoa butter, glyceryl behenate, glyceryl dipalmitostearate, and stearyl alcohol. Mixtures of mono-, di- and tri-glycerides and mono- and di-fatty acid esters of polyethylene glycol are also suitable fatty materials. Suitable waxes and wax-like materials include natural or synthetic waxes, hydrocarbons, and normal waxes. Specific examples of waxes include beeswax, glycowax, castor wax, carnauba wax, paraffins and candelilla wax.

Polyethylene glycol or its derivatives may include PEG 200, PEG 300, PEG 400, PEG 600, PEG 1000, PEG 4000, PEG 6000, PEG 8000, PEG 12000, PEG 20000, polyglycolyzed glycerides, polyethylene glycol-polyoxyethylenes, polyethylene glycol polypropylenes, and polyethylene glycol-polyoxypropylenes.

### Suspending/Thickening Agents

Suitable thickeners include, e.g., tragacanth; xanthan gum; bentonite; starch; acacia and lower alkyl ethers of cellulose (including the hydroxy and carboxy derivatives of the cellulose ethers). Examples of cellulose include, e.g., hydroxypropyl cellulose, hydroxypropyl methyl cellulose, sodium carboxy methylcellulose, microcrystalline cellulose (MCC), and MCC with sodium carboxyl methyl cellulose. In some embodiments, compositions according to the present disclosure include xanthan gum in an amount of from about 0.01 to about 1.0% weight per volume (w/v) of the composition, and more preferably about 0.1% w/v to about 0.2% w/v of the composition, e.g., about 0.15% w/v. In some embodiments, the compositions of the present disclosure include starch in an amount of about 1% w/v to about 10% w/v, e.g., about 1.5% w/v to about 2.5% w/v, e.g., about 2.0% w/v or about 2.1% w/v.

Suitable thickeners may also include diluents such as cellulose, dry starch, microcrystalline cellulose, dicalcium phosphate, calcium sulfate, sodium chloride, confectioner's sugar, compressible sugar, dextrates, dextrin, dextrose, sucrose, mannitol, powdered cellulose, sorbitol, and lactose.

### Binders

Binders may be used to impart cohesive qualities to compositions, and may include without limitation starch, gelatin, sugars, natural and synthetic gums, polyethylene glycol, ethylcellulose, methylcellulose, hydroxypropylmethylcellulose, carboxymethylcellulose, waxes and polyvinyl pyrrolidone.

### Humectants

The compositions of the present disclosure may also include a humectant. Suitable humectants useful in the finished formulations include glycerin, xylitol, polyethylene glycol, propylene glycol and mixtures thereof.

### Disintegrants

The compositions of the present disclosure may include a disintegrants to facilitate breakup or disintegration after administration. Materials used for this purpose include starches, clays, celluloses, aligns, gums, and cross-linked polymers.

### Surfactants

The compositions of the present disclosure may also comprise one or more surfactants in amounts of up to about 5.0% w/v and preferably from about 0.02 to about 3.0% w/v of the total formulation, e.g., about 0.1% w/v. The surfactants useful in the preparation of the finished compositions of the present disclosure are generally organic materials which aid in the stabilization and dispersion of the ingredients in aqueous systems for a suitable homogenous composition. Preferably, the surfactants of choice are non-ionic surfactants such as poly(oxyethylene) (20) sorbitan monooleate (available under the tradename TWEEN 80) and sorbitan monooleate. For example, in various embodiments, the compositions of the present disclosure include a poly(oxyethylene) (20) sorbitan monooleate in an amount of about 0.02 to about 3.0% w/v of the total formulation, e.g., about 0.1% w/v.

Exemplary non-ionic surfactants that can be included in the formulations of the present disclosure include, e.g., alkyl poly(ethylene oxide), alkyl polyglucosides (e.g., octyl glucoside and decyl maltoside), fatty alcohols such as cetyl alcohol and oleyl alcohol, cocamide MEA, cocamide DEA, and cocamide TEA. Specific non-ionic surfactants that can be included in the formulations produced by the present disclosure include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 188, poloxamer 407; polyethylene-polypropylene glycol; or polyethylene glycol (PEG).

### Antioxidants

The particles and compositions of the present disclosure may also comprise one or more antioxidants in amounts of up to about 5.0% w/v and preferably from about 0.02 to about 3.0% w/v of the total formulation, e.g., about 0.1% w/v. Suitable antioxidants include, but are not limited to ascorbic acid, sodium metabisulfite, sodium bisulfite, propyl gallate, butylated hydroxy anisole, butylated hydroxy toluene, citric acid, tartaric acid, phosphoric acid, tocopherol (vitamin E), sesamol and tertiary butyl hydroquinone. In some embodiments, the antioxidant is present in the Coated Granule 1 and 1.1-1.186 as described herein. In some embodiments, the antioxidant is present in the Composition 1 and 1.1-1.55 as described herein.

### Preservatives

The compositions of the present disclosure may include one or more preservatives. Useful preservatives include, but are not limited to, sodium benzoate, benzoic acid, potassium sorbate, salts of edetate (also known as salts of ethylenediaminetetraacetic acid, or EDTA, such as disodium EDTA), parabens (e.g., methyl, ethyl, propyl or butyl-hydroxybenzoates, etc.), and sorbic acid. Amongst useful preservatives include chelating agents some of which are listed above and other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3-Diaminopropanetetraacetic acid (1,3-PDTA); 2,2-ethylenedioxybis [ethyliminodi (acetic acid)] (EGTA); 1,10-bis(2-pyridylmethyl)-1,4,7,10-tetraazadecane (BPTETA); ethylenediamine (EDAMINE); Trans-1,2-diaminocyclohexane-N,N,N′,N′-tetraacetic acid (CDTA); ethylenediamine-N,N′-diacetate (EDDA); phenazine methosulphate (PMS); 2,6-Dichloro-indophenol (DCPIP); Bis(carboxymethyl) diaza-18-crown-6 (CROWN); porphine; chlorophyll; dimercaprol (2,3-Dimercapto-1-propanol); citric acid (e.g., citric acid anhydrous); tartaric acid; fumaric acid; malic acid; ascorbic acid; and salts thereof. The preservatives listed above are exemplary, but each preservative must be evaluated in each formulation, to assure the compatibility and efficacy of the preservative. Methods for evaluating the efficacy of preservatives in pharmaceutical formulations are known to those skilled in the art. Preferred preservatives are the paraben preservatives and include, methyl, ethyl, propyl, and butyl paraben, as well as benzoates, for example sodium benzoate. Methyl and propyl paraben, and sodium benzoate, are most preferable. Preferably, both methyl and propyl paraben are present in the formulation in a ratio of methyl paraben to propyl paraben of from about 2.5:1 to about 16:1, preferably 9:1. In certain embodiments, the composition includes parabens in an amount of about 0.01% w/v to about 0.5% w/v, e.g., about 0.1% w/v to about 0.3% w/v, e.g., about 0.2% w/v. In some embodiments, the compositions of the present disclosure include citric acid in an amount of about 0.001% w/v to about 0.1% w/v, e.g., about 0.01% w/v. In some embodiments, the composition includes ascorbic acid in an amount of about 0.001% w/v to about 0.1% w/v, e.g., about 0.06% w/v.

### Chelating Agents

As discussed above, suitable chelating agents include those listed above and other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3-Diaminopropanetetraacetic acid (1,3-PDTA); 2,2-ethylenedioxybis [ethyliminodi (acetic acid)] (EGTA); 1,10-bis(2-pyridylmethyl)-1,4,7,10-tetraazadecane (BPTETA); ethylenediamine (EDAMINE); Trans-1,2-diaminocyclohexane-N,N,N′,N′-tetraacetic acid (CDTA); ethylenediamine-N,N′-diacetate (EDDA).

### Buffering Agents

Suitable buffering agents include acetic acid and its salts, phosphoric acids and its salts; citric acid and its salts, and glutamic acid and its salts, as well as other buffering agents known to be useful in pharmaceutical compositions.

### Plasticizers

A plasticizer may be included in coating materials to alter their mechanical properties. Examples of plasticizers include benzyl benzoate, chlorobutanol, dibutyl sebacate, diethyl phthalate, glycerin, mineral oil, polyethylene glycol, sorbitol, triacetin, triethyl citrate, glycerol, etc. Further examples of plasticizers which may be used with the compositions of the present disclosure includes benzophenone, butyl phthalyl butyl glycollate, camphor, alpha-cresyl-p-toluene sulfonate, cyclohexyl-p-toluene sulfonamide, diamyl phthalate, dibutyl phthalate, dibutyl sebacate, dibutyl succinate, dibutyl tartrate, diethoxyethyl adipate, diethoxyethyl phthalate, diethyl adipate, diethylene glycol dipropionate, diethyl phthalate, diethyl sebacate, diethyl succinate, diethyl tartrate, dimethoxyethyl adipate, dimethoxyethyl phthalate, dimethyl phthalate, dipropyl phthalate, ethyl benzoyl benzoate, ethylene glycol diacetate, ethylene glycol dibutyrate, ethylene glycol dipropionate, ethyl phthalyl ethyl glycolate, methyl benzoyl benzoate, methyl phthalyl ethyl glycolate, o- or p-toluene ethyl sulfonamide, triacetin, tributyl citrate, tributyl phosphate, tributyrin, tricresyl phosphate, triethylene glycol diacetate, triethylene glycol dibutyrate, triethylene glycol diproprionate, triphenyl phosphate, tripropionin, trimellitates (e.g., tri-(2-ethylhexyl) trimellitate, tri-(isononyl) trimellitate, tri-(isodecyl) trimellitate, tri-(isotridecyl) trimellitate), adipates (e.g., bis(2-ethylhexyl) adipate), sebacates (e.g., dibutyl sebacate, di-(2-ethylhexyl) sebacate), glycerol triacetate, alkyl citrates (triethyl citrate, acetyl triethyl citrate, tributyl citrate, acetyl tributyl citrate, tri (2-ethylhexyl) citrate, acetyl trioctyl citrate, trihexyl citrate, butyryl trihexyl citrate), vegetable oils, epoxidized soybean oil, epoxidized linoleic oil, azelates, dibenzoates, terephthalates, 1,2-cyclohexane dicarboxylic acid diisononyl ester, alkyl sulphonic acid phenyl ester, organophosphates (e.g., tricresyl (methyl phenyl) phosphate, 2-ethylhexyldiphenyl phosphate), glycols (e.g., propylene glycol, polyethylene glycol, triethylene glycol di-2-hexanoate) triacetin, triethyl citrate, dibutyl sebacate, vegetable oil, lipids and combinations thereof.

### Colorants/Flavorants

The compositions of the present disclosure may include one or more flavorants. For example, the flavorant may comprise one or more sweeteners well known in the art, including both natural and artificial sweeteners. Thus, additional sweeteners may be chosen from the following non-limiting list: Water-soluble sweetening agents such as monosaccharides, disaccharides and polysaccharides such as xylose, ribose, glucose, mannose, galactose, fructose, high fructose corn syrup, dextrose, sucrose, sugar, maltose, partially hydrolyzed starch, or corn syrup solids and sugar alcohols such as sorbitol, xylitol, mannitol and mixtures thereof;

In general, the amount of sweetener may be between 0.001 to about 90% w/v of the final liquid composition, when using an easily extractable sweetener. The water-soluble sweeteners described above, are preferably used in amounts of about 5 to about 70% w/v, and most preferably from about 10 to about 50% w/v of the final liquid composition.

The flavorants included in the disclosed compositions may include mints such as peppermint, menthol, artificial vanilla, cinnamon, various fruit flavors, essential oils (i.e. thymol, eculyptol, menthol and methyl salicylate) and the like. The amount of flavoring employed is normally a matter of preference subject to such factors as flavor type, individual flavor, and strength desired. The flavorants are generally utilized in amounts that will vary depending upon the individual flavor, and may, for example, range in amounts of about 0.01 to about 3% by weight per volume of the final composition weight.

The colorants useful in the compositions of the present disclosure include pigments such as titanium dioxide, which may be incorporated in amounts of up to about 1% w/v, and preferably up to about 0.6% w/v. Also, the colorants may include dyes suitable for food, drug and cosmetic applications, and known as D&C and FD&C dyes and the like. The materials acceptable for the foregoing spectrum of use are preferably water-soluble. Illustrative examples include indigo dye, known as FD&C Blue No. 2, which is the disodium salt of 5,5′indigotindisulfonic acid. Similarly, the dye known as FD&C Green No. 1 comprises a triphenylmethane dye and is the monosodium salt of 4-[4-N-ethyl p-sulfobenzylamino) diphenylmethylene]-[1-(N-ethyl-N-p-sulfoniumbenzyl)-2,5-cyclohexadienimine]. Other such materials may be used as known to those skilled in the art.

### Administration Forms

In some embodiments, the coated drug-ion exchange resin particles and granules of the present disclosure are present in a form ready for administration, e.g., a blister pack, a bottle, syringes, foil packs, pouches, or other suitable container. In other embodiments, the compositions of the disclosure are in concentrated form in packs, optionally with the diluent required to make a final form for administration. In still other embodiments, the product contains a compound useful in the disclosure in solid form and, optionally, a separate container with a suitable suspension base or other carrier for the drug-ion exchange resin complex useful in the disclosure.

In still other embodiments, the above packs/kits include other components, e.g., a meter dose apparatus/device, instructions for dilution, mixing and/or administration of the product, other containers, etc. Other such pack/kit components will be readily apparent to one of ordinary skill in the art. In some preferred embodiments, the coated drug-ion exchange resin particles and granules of the present disclosure are present in a liquid suspension, as described herein.

### EXAMPLES

### Example 1—Preparation of Naltrexone Extended Release Suspension

Naltrexone can be prepared by various processes, for example those described in WO 91/05768, WO 2008/034973, WO 2008/138605, WO 2010/039209, and WO 2010/039209, the disclosures of each of which are incorporated by reference herein.

A Naltrexone resin complex was created according to the formulation shown in Table 1.

TABLE 1
Naltrexone Resin Complex

| Ingredient | Quantity |
|---|---|
| Naltrexone HCl | 0.5 kg |
| Amberlite IRP69 | 1.0 kg |
| Purified water* | 10.0 kg |

*Removed during process

Naltrexone can be prepared by various processes, for example those described in WO 91/05768, WO 2008/034973, WO 2008/138605, WO 2010/039209, and WO 2010/039209, the disclosures of each of which are incorporated by reference herein.

Purified water was dispensed into a container and naltrexone HCl was added and mixed until dissolved. Sodium polystyrene sulfonate (Amberlite IRP69 resin) was added to the tank and mixed. The resulting dispersion was passed through filter paper and the retained drug resin complex was washed with purified water. The drug resin complex was loaded into an oven for drying until the moisture was below 10%.

The formulation in Table 1 was used to create Naltrexone resin complex granules summarized below in Table 2. Polyethylene glycol was added into purified water and mixed until dissolved. The resulting PEG solution was sprayed on to the wet resin complex and granulated using a high shear granulator. The wet granules were dried in an oven until the moisture reaches between 15% to 20%. The partially dried granules were milled using a co-mill fitted with about 400-micron mesh screen. The pass-through granules were dried until the moisture reached below 7%.

TABLE 2
Composition of Naltrexone resin complex granules
Naltrexone resin complex granules

| Ingredient | Quantity |
|---|---|
| Naltrexone resin complex | 675 g |
| Polyethylene glycol | 122 g |
| Purified water* | 304 g |

*Removed during process

The resulting Naltrexone resin complex granules was coated to form the formulation summarized below in Table 3. Acetone and purified water were added to a container and mixed well. Polyethylene glycol was added to the container and mixed until dissolved. Cellulose acetate butyrate was added and mixed until dissolved. The coating solution was sprayed onto the naltrexone resin complex granules of Table 2 using a fluid bed processor to achieve a weight gain of 15%.

TABLE 3
Composition of Coated naltrexone resin
Coated naltrexone resin

| Ingredient | Quantity | |
|---|---|---|
| Naltrexone resin complex granules | 600 | g |
| Cellulose acetate butyrate | 78.9 | g |
| Polyethylene glycol | 15.8 | g |
| Acetone* | 1620 | g |
| Purified water* | 180 | g |

*Removed during process

The resulting coated naltrexone resin was then used to prepare a Naltrexone Extended-Release Suspension, summarized in Table 4 below. The suspension base was prepared by adding sucrose to the main container containing purified water and mixed until dissolved followed by adding citric acid. Starch was dispersed in the container and mixed well. In a separate container, methylparaben and propylparaben were added into propylene glycol and mixed until dissolved completely. Xanthan gum was added to the preservative solution and mixed well until it was uniformly dispersed. This gum dispersion was slowly added to the main container and mixed well. Sodium metabisulfite and Tween 80 were added into the main container and mixed until dissolved. Purified water was added to the main container to make up the final suspension base volume to 1.2 L. To 900 ml of the suspension base, coated naltrexone resin complex was added under continuous mixing to form the Naltrexone ER Suspension.

TABLE 4
Composition of Naltrexone Extended-Release Suspension

| | |
|---|---|
| Ingredient | Quantity |

Suspension base

| | | |
|---|---|---|
| Purified water | 650 | g |
| Sucrose | 240 | g |
| Citric acid anhydrous | 0.24 | g |
| Starch | 25.44 | g |
| Propylene glycol | 120 | g |
| Methylparaben | 2.16 | g |
| Propylparaben | 0.24 | g |
| Xanthan gum | 1.8 | g |
| Sodium metabisulfite | 1.2 | g |
| Tween 80 | 1.2 | g |

| | |
|---|---|
| Purified water | QS to 1.2 L |

Naltrexone Extended-Release suspension

| | | |
|---|---|---|
| Suspension base | 900 | mL |
| Coated naltrexone resin | 22.1 | g |

### Example 2—In-Vitro Drug Release

A coated naltrexone resin was prepared as per example 1. In this example, cherry flavor and FD&C red 40 were used to prepare the final suspension with ascorbic acid as an antioxidant.

TABLE 5
Naltrexone Extended-Release Suspension

| | |
|---|---|
| Ingredient | Quantity |

Suspension base

| | | |
|---|---|---|
| Purified water | 800 | g |
| Sucrose | 320 | g |
| Citric acid anhydrous | 0.16 | g |
| Starch | 33.92 | g |
| Propylene glycol | 160 | g |
| Methylparaben | 2.88 | g |
| Propylparaben | 0.32 | g |
| Xanthan gum | 2.4 | g |
| Ascorbic acid | 0.96 | g |
| Tween 80 | 1.6 | g |

| | |
|---|---|
| Purified water | QS 1600 mL |

Naltrexone ER Suspension

| | | |
|---|---|---|
| Suspension base | 500 | ml |
| Coated naltrexone resin (from example 1) | 12.29 | g |
| FD&C red 40 | 0.05 | g |
| Cherry flavor | 1.0 | g |

Purified water was added to a main container followed by sucrose and mixed until dissolved. Citric acid was added to the main container and mixed until dissolved. Starch was then dispersed in the container and mixed well. In a separate container, methylparaben and propylparaben were added into propylene glycol and mixed until dissolved completely. Xanthan gum was added to the preservative solution and mixed well until it was uniformly dispersed. The xanthan gum dispersion was slowly added to the main container and mixed well. Ascorbic acid and Tween 80 were added into the main container and mixed until dissolved. Purified water was added to the main container to make up the final suspension base volume to 1.6 L. To 500 mL of the suspension base, FD&C red number 40, Cherry flavor and coated naltrexone resin was added under continuous mixing to form the Naltrexone ER Suspension.

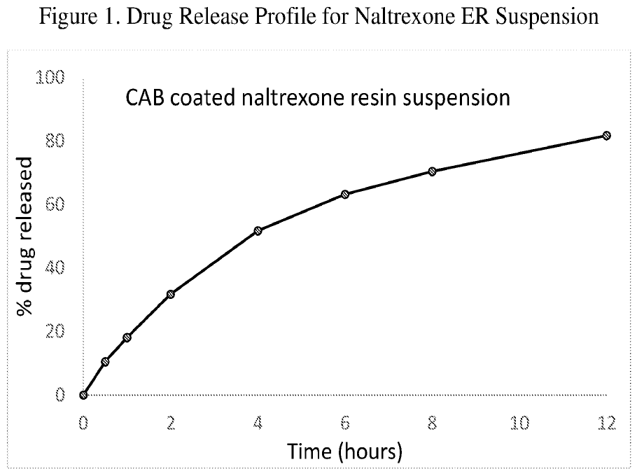

The Naltrexone ER Suspension was tested for in-vitro drug release using USP apparatus II, 0.4M KH₂PO₄, 900 mL, 50 rpm, 37±0.5° C. The results are summarized in FIG. 1.

### Example 3—Coated Naltrexone Resin

Naltrexone resin complex granules were prepared as per example 1. In this example cellulose acetate butyrate and hypromellose phthalate were used to coat the naltrexone resin complex granules. The formulation was prepared according to Table 6.

TABLE 6
Composition of Coated naltrexone resin

| Ingredient | Quantity | |
|---|---|---|
| Naltrexone resin complex granules | 500 | g |
| Cellulose acetate butyrate | 39 | g |
| Hypromellose phthalate | 39 | g |
| Acetone* | 1407.9 | g |
| Purified water* | 74.1 | g |

*Removed during process

To a container, acetone and purified water were added and mixed well. Polyethylene glycol was added to the container and mixed until dissolved. Cellulose acetate butyrate was added and mixed until dissolved. Hypromellose phthalate was then added and mixed until dissolved. The coating solution was sprayed on to naltrexone resin complex granules using fluid bed apparatus to achieve a weight gain of 15% w/w.

### Example 4—Coated Naltrexone Resin

Naltrexone resin complex granules were prepared as per example 1. In this example ethyl cellulose (EC) was used to coat the naltrexone resin complex granules. The formulation was prepared according to Table 7.

TABLE 7
Composition of Coated naltrexone resin

| Ingredient | Quantity | |
|---|---|---|
| Naltrexone resin complex granules | 500 | g |
| Ethyl cellulose | 65.7 | g |
| Myvacet 9-45 | 13.2 | g |
| Acetone* | 1349.5 | g |
| Purified water* | 150 | g |

*Removed during process

To a container, acetone and purified water were added and mixed well. Myvacet 9-45 was added and mixed until dissolved. Ethyl cellulose was added and mixed until dissolved. A total 1502 g of coating solution was sprayed onto the naltrexone resin complex granules using fluid bed processor to achieve a weight gain of 15% w/w.

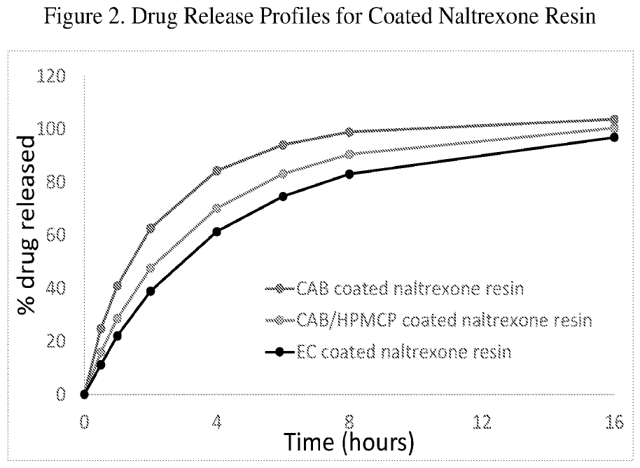

The coated naltrexone resin prepared as per example 4 was tested for in-vitro drug release using USP apparatus II, 0.4M KH₂PO₄, 900 mL, 50 rpm, 37±0.5° C. The results are summarized in FIG. 2.

### Example 5—Naltrexone Extended-Release Suspension

A Naltrexone resin complex was prepared according to the formulation shown in Table 8.

TABLE 8
Naltrexone resin complex

| Ingredient | Quantity |
|---|---|
| Naltrexone HCl | 0.5 kg |
| Amberlite IRP69 | 1.0 kg |
| Purified water* | 10.0 kg |

*Removed during process

Purified water was dispensed into a container and naltrexone HCl was added and mixed until dissolved. Sodium polystyrene sulfonate (Amberlite IRP69 resin) was added to the tank and mixed. The supernatant from the resulting dispersion was decanted followed by washing with purified water and finally the dispersion was filtered using a filter housing. The wet drug resin complex was dried using a fluid bed processor until the moisture content was about 15%.

The formulation in Table 8 was used to prepare Naltrexone resin complex granules summarized below in Table 9. EDTA was added into purified water and mixed until dissolved. Polyethylene glycol was added to the container and mixed until dissolved. The resulting PEG solution was sprayed on to the wet resin complex and granulated using a high shear granulator. The wet granules were dried using fluid bed processor until the moisture reaches between 15% to 20%. The partially dried granules were milled using a co-mill fitted with about 813-micron mesh screen. The pass-through granules were dried using fluid bed processor until the moisture reached below 7%.

TABLE 9
Composition of Naltrexone resin complex granules

| Ingredient | Quantity | |
|---|---|---|
| Naltrexone resin complex | 1300 | g |
| EDTA | 4 | g |
| Polyethylene glycol | 244 | g |
| Purified water* | 366 | g |

*Removed during process

The resulting complex was coated to form the formulation summarized below in Table 10. Acetone and purified water were added to a container and mixed well. Polyethylene glycol was added to the container and mixed until dissolved. Cellulose acetate butyrate was added and mixed until dissolved. The coating solution was sprayed onto the naltrexone resin complex granules of Table 9 using a fluid bed processor to achieve a weight gain of 15% w/w.

TABLE 10
Composition of Coated naltrexone resin

| Ingredient | Quantity | |
|---|---|---|
| Naltrexone resin complex granules | 500 | g |
| Cellulose acetate butyrate | 71 | g |
| Polyethylene glycol | 7.1 | g |
| Acetone* | 1424 | g |
| Purified water* | 75 | g |

*Removed during process

The resulting coated naltrexone resin was used to prepare a Naltrexone Extended-Release Suspension, summarized in Table 11 below. The suspension was prepared by adding glycerin to a container and heated to 45-60° C. Methylparaben and propylparaben were added to the container and mixed until dissolved. Xanthan gum was added to the preservative solution and mixed until it was uniformly dispersed (gum dispersion). To the main container containing purified water, sucrose, EDTA and citric acid were added and mixed until dissolved. Starch was added to the main container and mixed until uniformly dispersed. The gum dispersion was slowly added to the main container and mixed well. Tween 80, color and flavor were added to the main container and mixed well. The coated naltrexone resin was added to the main container and purified water was added to make up the final suspension volume to 1.6 L.

TABLE 11
Composition of Naltrexone Extended-Release Suspension

| Ingredient | Quantity | |
|---|---|---|
| Purified water | 676 | g |
| Sucrose | 320 | g |
| Citric acid anhydrous | 0.32 | g |
| EDTA | 1.6 | g |
| Starch | 33.9 | g |
| Glycerin | 240 | g |
| Methylparaben | 2.88 | g |
| Propylparaben | 0.32 | g |
| Xanthan gum | 2.4 | g |
| Tween 80 | 1.2 | g |
| Coated naltrexone resin | 38.46 | g |
| FD & C red 40 | 0.08 | g |
| Cherry flavor | 1.6 | g |

| | |
|---|---|
| Purified water | QS to 1.6 L |

### Example 6—Naltrexone Extended-Release Suspension

Coated naltrexone resin was prepared as per example 5. The naltrexone extended-release suspension was prepared as summarized in Table 12 below. The suspension was prepared by adding glycerin to a container and heated to 45-55° C. Methylparaben and propylparaben were added to the container and mixed until dissolved. Xanthan gum was added to the preservative solution and mixed until it was uniformly dispersed (gum dispersion). To the main container containing purified water, EDTA and citric acid were added and mixed until dissolved. Tween 80 was added and mixed well. Starch and sucrose were added together to the main container and mixed until uniformly dispersed. The gum dispersion was slowly added to the main container under continuous mixing. Color and flavor were added to the main container and mixed well. The coated naltrexone resin was added to the main container and purified water was added to make up the final suspension volume to 1.6 L.

TABLE 12
Composition of Naltrexone Extended-Release Suspension

| Ingredient | Quantity | |
|---|---|---|
| Purified water | 680 | g |
| Sucrose | 320 | g |
| Citric acid anhydrous | 0.32 | g |
| EDTA | 1.6 | g |
| Starch | 33.9 | g |
| Glycerin | 960 | g |
| Methylparaben | 2.88 | g |
| Propylparaben | 0.32 | g |
| Xanthan gum | 2.4 | g |
| Tween 80 | 1.2 | g |
| Coated naltrexone resin | 38.46 | g |
| FD & C red 40 | 0.08 | g |
| Cherry flavor | 1.6 | g |

| | |
|---|---|
| Purified water | QS to 1.6 L |

### Example 7—Preparation of Naltrexone Extended-Release Suspension

A Naltrexone resin complex was prepared according to the formulation shown in Table 13.

TABLE 13
Naltrexone resin complex

| Ingredient | Quantity (kg) |
|---|---|
| Naltrexone HCl | 1.5 |
| Amberlite IRP69 | 3.0 |
| Purified water* | 30.0 |

*Removed during process

Purified water was dispensed into a container and naltrexone HCl was added and mixed until dissolved. Sodium polystyrene sulfonate (Amberlite IRP69 resin) was added to the tank and mixed. The supernatant from the resulting dispersion was decanted followed by washing with purified water and finally the dispersion was filtered using a filter housing. The wet drug resin complex was dried using a fluid bed processor until the moisture content was about 15%.

The formulation in Table 13 was used to prepare Naltrexone resin complex granules summarized below in Table 14. EDTA was added into purified water and mixed until dissolved. Polyethylene glycol (PEG) was added to the container and mixed until dissolved. PEG solution was sprayed on to the wet resin complex and granulated using a high shear granulator. The wet granules were dried using fluid bed processor until the moisture reaches between 15% to 25%. The partially dried granules were passed through #20 mesh screen and dried using the fluid bed dryer until the moisture reached below 7%. These dried granules were milled using a co-mill fitted with 813-micron mesh screen.

TABLE 14
Composition of Naltrexone resin complex granules

| Ingredient | Quantity (g) |
|---|---|
| Naltrexone resin complex | 4300 |
| EDTA | 16 |
| Polyethylene glycol | 946 |
| Purified water* | 1.42 |

*Removed during process

The resulting complex was coated to different weight gains using the coating composition shown in Table 15. Acetone and purified water were added to a container and mixed well. Polyethylene glycol was added to the container and mixed until dissolved. Cellulose acetate butyrate was added and mixed until dissolved. The coating solution was sprayed onto the naltrexone resin complex granules of Table 14 using a fluid bed processor to achieve a coating weight gain of 15% w/w and 20% w/w.

TABLE 15
Composition of Coating Solution

| Ingredient | Quantity (g) |
|---|---|
| Cellulose acetate butyrate | 96 |
| Polyethylene glycol | 9.6 |
| Acetone* | 1906.1 |
| Purified water* | 100.3 |

*Removed during process

The resulting coated naltrexone resin was used to prepare a Naltrexone Extended-Release Suspension, summarized in Table 16 below. To the main container containing purified water, Tween 80, propyl gallate, sodium benzoate, EDTA and citric acid anhydrous were added and mixed until dissolved. Xanthan gum, starch and sucrose were mixed together and added into the main tank and mixed until uniformly dispersed. Color solution was added into the main tank while mixing. The coated naltrexone resin and flavor was added to the main container and purified water was added to make up the final suspension volume to 4 L.

TABLE 16
Composition of Naltrexone Extended-Release Suspension

| | |
|---|---|
| Formulation 1 | Formulation 2 |

| | |
|---|---|
| Ingredient | Quantity (g) |

| | | |
|---|---|---|
| Purified water | 2680 | 2680 |
| Sucrose | 800 | 800 |
| Citric acid anhydrous | 16 | 16 |
| EDTA | 3.8 | 3.8 |
| Starch | 84.39 | 84.39 |
| Sodium benzoate | 8 | 8 |
| Xanthan gum | 6 | 6 |
| Tween 80 | 4 | 4 |
| Propyl gallate | 0.8 | 0.8 |
| Coated naltrexone resin | 85.1 | 89.3 |
| FD & C red 40 | 0.2 | 0.2 |
| Cherry flavor | 4 | 4 |
| Purified water | QS to 4 L | QS to 4 L |

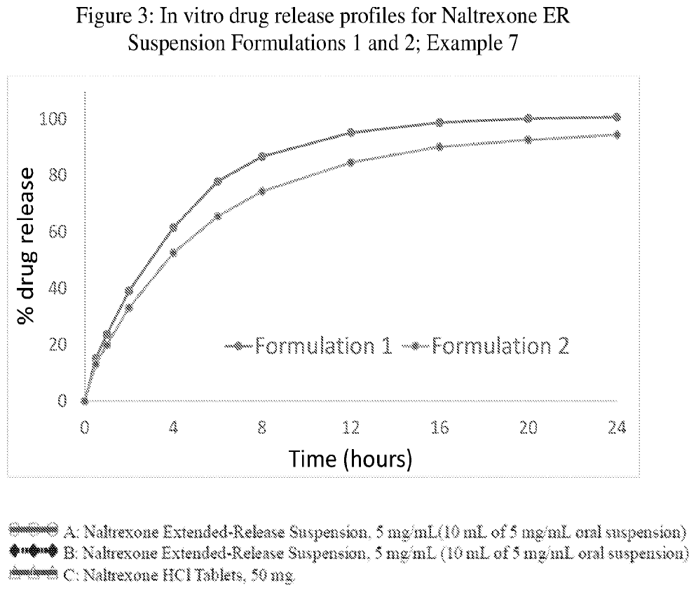

FIG. 3 shows the in vitro drug release profiles for the Naltrexone Extended-Release Suspension Formulations 1 and 2 described in Table 16.

### Pharmacokinetic Study:

Study outline: The study was an open-label, single-dose, randomized, three-period, three-treatment, three-sequence, crossover, comparative bioavailability study in healthy adults (n=18) under fasted condition.

### Treatments:

Treatment A—Formulation 1:10 mL of 5 mg/mL oral suspension (Total dose=50 mg naltrexone HCl) administered after an overnight fast of at least 10 hours.
Treatment B—Formulation 2:10 mL of 5 mg/mL oral suspension (Total dose=50 mg naltrexone HCl) administered after an overnight fast of at least 10 hours.
Treatment C—Reference product: Naltrexone HCl Tablets, 50 mg (Mallinckrodt™, SpecGx LLC): one (1) 50 mg tablet administered after an overnight fast of at least 10 hours.

Analytes: Both naltrexone and its active metabolite (6-β-Naltrexol) were analyzed

### Results:

The AUCₜ, AUC_{∞}, Cₘₐₓ, Tₘₐₓ and T_{1/2} values obtained for Naltrexone and 6-β-Naltrexol are shown in Table 17:

TABLE 17
Pharmacokinetic Parameters

| | | | | | |
|---|---|---|---|---|---|
| | AUCₜ | AUC_{∞} | Cₘₐₓ | Tₘₐₓ | T_{1/2} |
| Product | (hr · ng/mL) | (hr · ng/mL) | (ng/mL) | (hrs) | (hrs) |

Naltrexone

| | | | | | |
|---|---|---|---|---|---|
| Formulation 1 (A) | 29.705 | 31.762 | 2.904 | 5.91 | 7.07 |
| Formulation 2 (B) | 28.211 | 32.450 | 2.088 | 5.75 | 11.10 |
| Reference (C) | 20.859 | 21.989 | 8.199 | 0.78 | 5.44 |

6-β-Naltrexol

| | | | | | |
|---|---|---|---|---|---|
| Formulation 1 (A) | 790.770 | 905.573 | 34.871 | 6.21 | 14.84 |
| Formulation 2 (B) | 690.615 | 861.430 | 24.078 | 8.97 | 16.42 |
| Reference (C) | 779.762 | 905.573 | 102.753 | 0.91 | 14.01 |

The Mean Plasma Naltrexone and Mean Plasma 6-B-Naltrexol Concentrations are shown in Tables 18 and 19, respectively:

TABLE 18
Mean Plasma Naltrexone Concentrations

| | Formulation 1 | Formulation 2 | Reference |
|---|---|---|---|
| Time (hrs) | (ng/mL) | (ng/mL) | (ng/mL) |
| 0 | 0.000 | 0.000 | 0.000 |
| 0.25 | 0.247 | 0.217 | 1.313 |
| 0.5 | 0.741 | 0.679 | 5.855 |
| 0.75 | 0.849 | 0.722 | 6.712 |
| 1 | 0.834 | 0.733 | 6.257 |
| 1.5 | 0.977 | 0.808 | 4.734 |
| 2 | 1.160 | 0.876 | 3.758 |
| 2.5 | 1.336 | 0.957 | 3.129 |
| 3 | 1.429 | 0.968 | 2.475 |
| 3.5 | 1.467 | 0.997 | 2.036 |
| 4 | 1.495 | 1.020 | 1.679 |
| 6 | 2.833 | 2.019 | 0.843 |
| 8 | 1.952 | 1.570 | 0.455 |
| 10 | 1.552 | 1.340 | 0.391 |
| 12 | 1.319 | 1.177 | 0.325 |
| 16 | 0.771 | 0.845 | 0.177 |
| 24 | 0.312 | 0.486 | 0.038 |
| 36 | 0.088 | 0.180 | 0.000 |
| 48 | 0.021 | 0.047 | 0.000 |

TABLE 19
Mean Plasma 6-β-Naltrexol Concentrations

| | Formulation 1 | Formulation 2 | Reference |
|---|---|---|---|
| Time (hrs) | (ng/mL) | (ng/mL) | (ng/mL) |
| 0 | 0.000 | 0.000 | 0.000 |
| 0.25 | 2.454 | 2.121 | 12.150 |
| 0.5 | 7.811 | 6.604 | 68.629 |
| 0.75 | 10.381 | 8.384 | 91.124 |
| 1 | 11.135 | 8.885 | 90.165 |
| 1.5 | 13.860 | 10.644 | 74.653 |
| 2 | 17.764 | 12.105 | 66.465 |
| 2.5 | 20.812 | 13.924 | 59.188 |
| 3 | 22.888 | 14.713 | 53.147 |
| 3.5 | 24.649 | 16.239 | 47.947 |
| 4 | 26.512 | 17.596 | 42.929 |
| 6 | 33.771 | 22.778 | 33.900 |
| 8 | 30.129 | 21.856 | 25.441 |
| 10 | 27.771 | 21.511 | 21.535 |
| 12 | 27.088 | 21.312 | 19.124 |
| 16 | 23.094 | 20.046 | 15.640 |
| 24 | 17.384 | 17.689 | 10.878 |
| 36 | 8.346 | 9.344 | 5.272 |
| 48 | 5.182 | 5.678 | 3.458 |

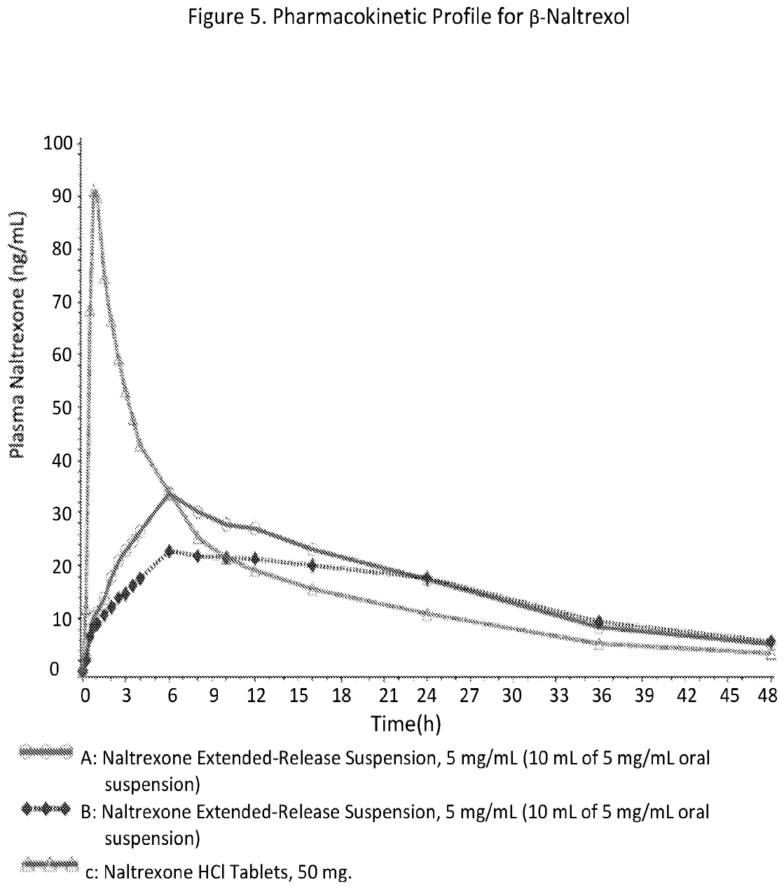

The Pharmacokinetic Profile for Naltrexone Formulations 1 and 2, and the commercial product (Naltrexone HCl Tablets, 50 mg; Mallinckrodt™, SpecGx LLC) is shown in FIG. 4. The Pharmacokinetic Profile for 6-B-Naltrexol Formulations and the same commercial product is shown in FIG. 5.

### Study Findings:

The results show overall that the Formulations of the present disclosure displayed better bioavailability (˜50% more bioavailable) compared to the commercial immediate release tablet. For naltrexone there was an ˜65% reduction in Cₘₐₓ for Formulation 1 and ˜75% reduction in Cₘₐₓ for Formulation 2. This significantly reduces side effects caused by abrupt rise of plasma concentration from immediate release product.

The Tₘₐₓ was delayed from less than 1 hour for reference intermediate release (IR) tablet to ˜6 hours for both Formulations 1 & 2, showing the extended-release nature of the test products. The duration of effectiveness for the Formulations 1 & 2 was prolonged compared to immediate release reference product due to slower rise and drop of plasma concentration. Formulations 1 & 2 maintain plasma concentration above 0.75 ng/ml for ˜16 hours after oral ingestion, however, it is only ˜6 hours for the reference product.

For 6-β-Naltrexol there was a ˜66% reduction in Cₘₐₓ for Formulation 1 and ˜77% reduction in Cₘₐₓ for Formulation 2. This significantly reduces side effects caused by abrupt rise of plasma concentration from immediate release product. Similar to the pattern for naltrexone's plasma profile, the active metabolite, 6-β-Naltrexol, derived from Formulations 1 & 2 exhibited lower Cₘₐₓ and longer Tₘₐₓ compared to reference product. However, comparable bioavailability was observed for Formulations 1 and 2, and the reference product.

The biological half-life for 6-β-Naltrexol is comparable among Formulations 1 & 2 and the reference product. The plasma profile of the extended-release Formulations 1 & 2 exhibit higher concentration than the reference product from 10 hours onward after oral ingestion.

While the invention has been described in connection with what is presently considered to be the most practical and preferred embodiment, it is to be understood that the invention is not to be limited to the disclosed embodiments, but to the contrary, it is intended to cover various modifications or equivalent arrangements included within the spirit and scope of the appended claims. The scope is to be accorded the broadest interpretation so as to encompass all such modifications and equivalent structures as is permitted under the law.

Each of the patents, books, articles and other printed publications referenced herein are incorporated by reference in their entireties for all purposes.

1. A coated drug-ion exchange resin particle comprising:
a) a drug-ion exchange resin complex (or resinous core) comprising:
an ion exchange resin;
one or more active pharmaceutical agents;
optionally a chelating agent;
optionally a granulating agent; and
b) a coating comprising:
a cellulose acetate-containing polymer;
optionally, an enteric polymer; and
optionally, a plasticizer.

2. The coated drug-ion exchange resin particle according to claim 1, wherein the ion exchange resin comprises a co-polymer of polystyrene and divinylbenzene; for example Amberlite IRP-69.

3. The coated drug-ion exchange resin particle according to claim 1, wherein the cellulose acetate polymer is selected from cellulose acetate, cellulose diacetate, cellulose triacetate, cellulose acetate butyrate, cellulose acetate phthalate, cellulose acetate propionate, and combinations thereof; for example cellulose acetate butyrate.

4. The coated drug-ion exchange resin particle according to claim 1, wherein the enteric polymer is selected from a phthalate-containing polymer; cellulose acetate trimellitate; polymethacrylates; hydroxypropylmethylcellulose acetate succinate (HPMC AS; Aqoat®); sodium alginate; alginic acid; shellac and combinations thereof; for example one or more of cellulose acetate phthalate (CAP), hypromellose phthalate (hydroxypropylmethylcellulose phthalate; HPMCP) and polyvinyl acetate phthalate (PVAP).

5. The coated drug-ion exchange resin particle according to claim 1, wherein the chelating agent, granulating agent and plasticizer are each present.

6. The coated drug-ion exchange resin particle according to claim 5, wherein the chelating agent comprises EDTA; the granulating agent comprises polyethylene glycol;
and the plasticizer comprises polyethylene glycol.

7. The coated drug-ion exchange resin particle according to claim 1, wherein the active pharmaceutical agent is present in an amount of about 1% to about 99% by weight based on the total weight of the coated drug-ion exchange resin granule, from about 5% to about 70% by weight, from about 10% to about 30% by weight, from about 15% to about 25% by weight, or about 20% by weight based on the total weight of the coated drug ion exchange resin granule.

8. The coated drug-ion exchange resin particle according to claim 7, having the following composition:

| Ingredient | % w/w |
|---|---|
| Drug-ion exchange resin granules (or | 70-90; e.g. 75-90; e.g. 80- |
| resinous core) | 85; e.g. 83-84; e.g. 83.3 |
| Cellulose acetate-containing polymer, e.g. | 5-25; e.g. 10-20; e.g. 12- |
| cellulose acetate butyrate | 18; e.g. 14-16%; e.g. 15- |
| | 15.4; e.g. 15.2 |
| Plasticizer, e.g. polyethylene glycol (PEG), | 0.5-5; e.g. 0.5-3; e.g. 1-2; |
| e.g. PEG 3350 | e.g. 1.5 |

9. A composition for sustained release of an active pharmaceutical agent, said composition comprising:
a plurality of coated drug-ion exchange resin particles (or granules) according to claim 1; and
a pharmaceutically acceptable excipient;
wherein the composition is in the form of a liquid suspension and further comprises one or more solvents, and one or more ingredients selected from sugars, antioxidants, buffering agents, chelating agents, thickeners, stabilizers, preservatives, surfactants, flavorings and coloring agents.

10-22. (canceled)

23. The composition for sustained release according to claim 9, wherein:
the coated granules are present in the composition in an amount of about 0.01% w/v to about 10% w/v, based on the total weight of the composition; or about 0.01% w/v to about 10% w/v, based on the total weight of the composition; or about 0.1% w/v to about 5% w/v, based on the total weight of the composition; or about 0.1% w/v to about 5% w/v, based on the total weight of the composition; or about 1% w/v to about 5% w/v, based on the total weight of the composition; or about 1% w/v to about 5% w/v, based on the total weight of the composition; or about 2% w/v to about 3% w/v, based on the total weight of the composition; or about 2% w/v to about 3% w/v, based on the total weight of the composition;
the active pharmaceutical agent is present in an amount from about 0.1 mg/mL to about 100 mg/mL, e.g., from about 1 mg/mL to about 25 mg/mL, e.g., from about 3 mg/mL to about 8 mg/mL, e.g., about 5 mg/mL;
and the composition further comprises:
a chelating agent, for example EDTA;
a buffering agent, e.g. citric acid;
a sugar;
one or more thickeners;
a preservative;
a surfactant; and
an antioxidant.

24. The composition for sustained release according to claim 9, having the following ingredients:

| Ingredient | Quantity (g) | % w/v |
|---|---|---|
| Sugar (e.g. Sucrose) | 600-1000; e.g. 800 | 5-40; e.g. 10- |
| | | 30%; e.g. 15%- |
| | | 25%; e.g. 20 |
| Buffering agent (e.g. | 10-20; e.g. 14-18; e.g. | 0.1-0.8; e.g. 0.2- |
| citric acid anhydrous) | 16 | 0.6; e.g. 0.25-0.5; |
| | | e.g. 0.3-0.5; e.g. |
| | | 0.35-0.45; e.g. 0.4 |
| Chelating agent (e.g. | 2-6; e.g. 3-5; e.g. 3.5- | 0.05-0.15; e.g. |
| EDTA) | 4.5; e.g., 3.8 | 0.075-0.125; e.g. |
| | | 0.08-0.12; e.g. |
| | | 0.09-0.10; e.g. |
| | | 0.095 |
| Thickener (e.g. Starch) | 70-100; e.g. 80-90; | 0.1-5; e.g. 1-3; |
| | e.g. 84-85, e.g. 84.39 | e.g. 1.75-2.5; e.g. |
| | | 2.0-2.25; e.g. 2.1- |
| | | 2.3; e.g. 2; e.g., |
| | | 2.1 |
| Preservative (e.g. Sodium | 4-12; e.g. 6-10; e.g. 8 | 0.1-0.5; e.g. 0.1- |
| benzoate) | | 0.3; e.g. 0.15- |
| | | 0.25; e.g. 0.2 |
| Thickener (e.g. Xanthan | 2-10; e.g. 4-8; e.g. | 0.05-0.5; e.g. 0.1- |
| gum) | 5-7; e.g. 6 | 0.2; e.g. 0.15 |
| Surfactant (e.g. Tween 80) | 2-6; e.g. 3-5; e.g. 4 | 0.01-0.3; e.g. |
| | | 0.05-0.2; e.g. 0.1 |
| Anti-oxidant (e.g. propyl | 0.4-1.2; e.g. 0.6-1.0; | 0.01-0.03; e.g. |
| gallate) | e.g. 0.8 | 0.02 |
| Coated Granules | 60-110; e.g. 70-100; | 0.5-5; e.g. 1.0- |
| | e.g., 80-90; e.g. 85; | 3.0; e.g. 2.0-2.5; |
| | e.g., 89 | e.g. 2.3; e.g. about |
| | | 2.28 |
| Color agent (e.g. FD & C | 0.1-1.3; e.g. 0.2 | 0.005 |
| red 40) | | |
| Flavor (e.g. Cherry | 2-6; e.g. 4 | 0.1 |
| flavor) | | |
| Purified water | QS to 4 L | QS to 4 L |

25. The composition for sustained release according to claim 9, wherein no more than 25% or 30% of the active pharmaceutical agent is released within one hour; and wherein no more than 60% of the active pharmaceutical agent is released within two hours.

26. The composition for sustained release according to claim 9, wherein the active pharmaceutical agent is released at a higher initial rate that tapers over a sustained period of time, and the composition provides:
sustained release of the active pharmaceutical agent for at least 1 hour, at least 4 hours, at least 8 hours, at least 16 hours, or at least 24 hours; and/or
sustained release of the active pharmaceutical agent for up to 24 hours; and/or
sustained release of the active pharmaceutical agent for 8-24 hours.

27. A method for preparing a coated drug-ion exchange resin particle comprising:
(a) contacting an active drug substance and an ion exchange resin in a solvent to form a drug-ion exchange resin complex (or resinous core);
(b) optionally isolating and drying the drug-ion exchange resin complex;
(c) granulating the drug-ion exchange resin complex to form a drug-ion exchange resin complex granule;
(d) optionally drying the product of step (c), for example to 15-25% or 15-20% moisture content, and optionally milling the product, for example using a Comil fitted with about 400-micron mesh screen; or optionally screening the product to a predetermined size, for example by passing the product through a mesh screen;
(e) optionally further drying the product of step (d), for example to <7% moisture content;
(f) optionally milling the product of step (e), for example with a Comil fitted with a mesh screen; and
(g) coating the product of the preceding steps with a coating composition comprising:
a cellulose acetate-containing polymer, for example cellulose acetate butyrate;
optionally, an enteric polymer;
optionally, a plasticizer, e.g., polyethylene glycol, e.g. polyethylene glycol 3550; and
a solvent for example water, acetone, or water and acetone;
for example wherein the coating is applied to a weight gain of 15-20% w/w;
to form the coated drug-ion exchange resin particle (or granule).

28. The method of claim 27, wherein the coated drug-ion exchange resin particle produced by the method is a coated drug-ion exchange resin particle comprising:
a) a drug-ion exchange resin complex (or resinous core) comprising:
an ion exchange resin;
one or more active pharmaceutical agents;
optionally a chelating agent;
optionally a granulating agent; and
b) a coating comprising:
a cellulose acetate containing polymer;
optionally, an enteric polymer; and
optionally, a plasticizer.

29. The method of claim 27, further comprising the step of:
h) combining the coated drug-ion exchange resin particle (or granule) produced by step (g) and a suspension base composition to form a coated drug-ion exchange resin extended-release suspension;
wherein the suspension base composition is in the form of a liquid suspension, and comprises one or more solvents, and one or more ingredients selected from sugars, antioxidants, buffering agents, chelating agents, thickeners, stabilizers, preservatives, surfactants, flavorings and coloring agents.

30. A method for preparing a coated drug-ion exchange resin particle comprising:
(a) contacting an active drug substance and an ion exchange resin in a solvent to form a drug-ion exchange resin complex (or resinous core);
(b) optionally isolating and drying the drug-ion exchange resin complex;
(c) granulating the drug-ion exchange resin complex to form a drug-ion exchange resin complex granule;
(d) optionally drying the product of step (c), for example to 15-25% or 15-20% moisture content, and optionally milling the product, for example using a Comil fitted with about 400-micron mesh screen; or optionally screening the product to a predetermined size, for example by passing the product through a mesh screen;
(e) optionally further drying the product of step (d), for example to <7% moisture content;
(f) optionally milling the product of step (e), for example with a Comil fitted with a mesh screen; and
(g) coating the product of the preceding steps with a coating composition comprising:
a cellulose acetate-containing polymer, for example cellulose acetate butyrate;
optionally, an enteric polymer;
optionally, a plasticizer, e.g., polyethylene glycol, e.g. polyethylene glycol 3550; and
a solvent for example water, acetone, or water and acetone;
for example wherein the coating is applied to a weight gain of 15-20% w/w;
h) combining the coated drug-ion exchange resin particle (or granule) produced by step (g) and a suspension base composition to form a coated drug-ion exchange resin extended-release suspension;
wherein the suspension base composition is in the form of a liquid suspension, and comprises one or more solvents, and one or more ingredients selected from sugars, antioxidants, buffering agents, chelating agents, thickeners, stabilizers, preservatives, surfactants, flavorings and coloring agents;
to form the coated drug-ion exchange resin particle (or granule);
wherein the composition formed by the method is a composition according to claim 9.

31. A liquid extended-release oral naltrexone suspension formulation comprising: naltrexone or a pharmaceutically acceptable salt thereof in an amount of about 1.0 mg/mL to about 10.0 mg/mL, wherein the naltrexone or a pharmaceutically acceptable salt thereof is bound to a resin, for example an ion exchange resin;
wherein the formulation provides an in vivo pharmacokinetic profile having one or more of the following characteristics a-e:
a) Tₘₐₓ of about 5 to about 7 hours, for example about 5 hours, about 6 hours or about 7 hours;
b) T_{1/2} of about 5 to about 12 hours, for example about 5 to about 11 hours; about 6 hours, about 7 hours, about 8 hours, about 9 hours, about 10 hours or about 11 hours;
c) Cₘₐₓ of about 1.5 to about 4 hours, for example about 1.5 hours, about 2 hours about 2.5 hours, about 3 hours, about 3.5 hours or about 4 hours;
d) AUCₜ of about 26 to about 32 hr·ng/mL, for example about 26, about 27, about 28, about 29, about 30, about 31 or about 32 hr·ng/mL; and
e) AUCₜ of about 28 to about 35 hr·ng/mL, for example about 28, about 29, about 30, about 31, about 32, about 33, about 34 of about 35 hr·ng/mL.

32. The liquid extended-release oral naltrexone suspension formulation of claim 31, wherein the formulation provides an in vivo pharmacokinetic profile having two, three, four or all five of said characteristics a-e.

33. The liquid extended-release oral naltrexone suspension formulation of claim 31, wherein the in vivo pharmacokinetic profile is substantially in accordance with Formulation 1 or Formulation 2 in FIG. 4.

34. The liquid extended-release oral naltrexone suspension formulation of claim 31, wherein the ratio of the Mean Plasma Naltrexone Concentration (Cₜ, ng/mL) to the Maximum Plasma Naltrexone Concentration (Cₘₐₓ; ng/mL) is one or more of a-e below:
a) three hours after administration, from about 0.4 to about 0.6; for example, from about 0.4 to about 0.55, for example from about 0.45 to about 0.5;
b) six hours after administration, from about 0.8 to about 1.2; for example, from about 0.9 to about 1.1, for example from about 0.95 to about 1.05;
c) eight hours after administration, from about 0.5 to about 0.9; for example, from about 0.6 to about 0.8, for example from about 0.65 to about 0.8;
d) ten hours after administration, from about 0.4 to about 0.8; for example, from about 0.5 to about 0.7; and
e) twelve hours after administration, from about 0.3 to about 0.7; for example, from about 0.4 to about 0.6.

35. A composition for sustained release of naltrexone, said composition comprising: a plurality of coated drug-ion exchange resin particles (or granules) comprising:
a) a drug-ion exchange resin complex (or resinous core) comprising:
an ion exchange resin;
naltrexone or a pharmaceutically acceptable salt thereof;
optionally a chelating agent;
optionally a granulating agent; and
b) a coating comprising:
a cellulose acetate-containing polymer;
optionally, an enteric polymer; and
optionally, a plasticizer;
and
a pharmaceutically acceptable excipient;
wherein the composition is in the form of a liquid suspension and further comprises one or more solvents, and one or more ingredients selected from sugars, antioxidants, chelating agents, thickeners, stabilizers, preservatives, surfactants, flavorings and coloring agents;
wherein the formulation provides an in vivo pharmacokinetic profile having one or more of the following characteristics a-e:
a) Tₘₐₓ of about 5 to about 7 hours, for example about 5 hours, about 6 hours or about 7 hours;
b) T_{1/2} of about 5 to about 12 hours, for example about 5 to about 11 hours; about 6 hours, about 7 hours, about 8 hours, about 9 hours, about 10 hours or about 11 hours;
c) Cₘₐₓ of about 1.5 to about 4 hours, for example about 1.5 hours, about 2 hours about 2.5 hours, about 3 hours, about 3.5 hours or about 4 hours;
d) AUCₜ of about 26 to about 32 hr·ng/mL, for example about 26, about 27, about 28, about 29, about 30, about 31 or about 32 hr·ng/mL; and
c) AUC_{∞} of about 28 to about 35 hr·ng/mL, for example about 28, about 29, about 30, about 31, about 32, about 33, about 34 of about 35 hr·ng/mL.

36. The composition for sustained release according to claim 35, wherein the ratio of the Mean Plasma Naltrexone Concentration (Cₜ; ng/ml) to the Maximum Plasma Naltrexone Concentration (Cₘₐₓ; ng/mL) is one or more of a-e below:
a) three hours after administration, from about 0.4 to about 0.6; for example, from about 0.4 to about 0.55, for example from about 0.45 to about 0.5;
b) six hours after administration, from about 0.8 to about 1.2; for example, from about 0.9 to about 1.1, for example from about 0.95 to about 1.05;
c) eight hours after administration, from about 0.5 to about 0.9; for example, from about 0.6 to about 0.8, for example from about 0.65 to about 0.8;
d) ten hours after administration, from about 0.4 to about 0.8; for example, from about 0.5 to about 0.7; and
e) twelve hours after administration, from about 0.3 to about 0.7; for example, from about 0.4 to about 0.6.
